(19) United States
(12) Patent Application Publication
(10) Pub. No.: US 2006/0247292 A1
(43) Pub. Date: Nov. 2, 2006

(54) Benzocycloheptene derivatives, process for their production and their use as anti-inflammatory agents

(76) Inventors: Hartmut Rehwinkel, Berlin (DE); Duy Nguyen, Berlin (DE); Markus Berger, Berlin (DE); Heike Schaecke, Berlin (DE)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 11/384,809

(22) Filed: Mar. 21, 2006

Related U.S. Application Data
(60) Provisional application No. 60/669,885, filed on Apr. 11, 2005.

(30) Foreign Application Priority Data
Mar. 22, 2005 (DE) 10 2005 014 090.4

(51) Int. Cl. A61K 31/415

(52) U.S. Cl. 514/406

(57) ABSTRACT

The invention relates to polysubstituted 5H-benzocycloheptene derivatives of formula (I) 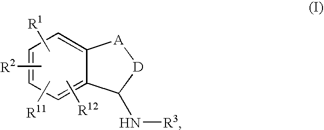 process for their production, and their use as anti-inflammatory agents.

The invention relates to 5H-benzocycloheptene derivatives, process for their production and their use as anti-inflammatory agents.

Open-chain, non-steroidal anti-inflammatory agents are known from the prior art WO 00/32584, DE 100 38 639 and WO02/10143. In the experiment, these compounds show dissociations of action between anti-inflammatory and undesirable metabolic actions and are superior to the previously described, non-steroidal glucocorticoids or exhibit at least just as good an action.

Since the compounds of the prior art, however, do not always have undesirable side effects or are not selective enough, in addition the object exists to make available non-steroidal anti-inflammatory agents whose profile is improved relative to the compounds of the prior art.

This object is achieved by the compounds of this invention, explained in the claims.

This invention therefore relates to compounds of general formula (I), 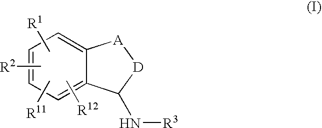
in which
R¹ and R², independently of one another, mean a hydrogen atom, a hydroxy group, a halogen atom, an optionally substituted (C₁-C₁₀)-alkyl group, a (C₁-C₁₀)-alkoxy group, a (C₁-C₁₀)-alkylthio group, a (C₁-C₅)-perfluoro-alkyl group, a cyano group, or a nitro group, or
R¹ and R² together mean a group that is selected from the groups —O—(CH₂)ₙ—O—, —O—(CH₂)ₙ—CH₂—, —O—CH═CH—, —(CH₂)ₙ₊₂—, —NH—(CH₂)ₙ₊₁—, —N(C₁-C₃-alkyl)-(CH₂)ₙ₊₁—, and —NH—N═CH—,
whereby n=1 or 2, and the terminal oxygen atoms are linked to directly adjacent ring-carbon atoms, or NR⁸R⁹, whereby R⁸ and R⁹, independently of one another, can be hydrogen, C₁-C₅-alkyl or (CO)-C₁-C₅-alkyl,
R¹¹ means a hydrogen atom, a hydroxy group, a halogen atom, a cyano group, an optionally substituted (C₁-C₁₀)-alkyl group, a (C₁-C₁₀)-alkoxy group, a (C₁-C₁₀)-alkylthio group, or a (C₁-C₅)-perfluoroalkyl group,
R¹² means a hydrogen atom, a hydroxy group, a halogen atom, a cyano group, an optionally substituted (C₁-C₁₀)-alkyl group, or a (C₁-C₁₀)-alkoxy group,
R³ means a C₁-C₁₀-alkyl group, which optionally can be substituted by a group that is selected from 1-3 hydroxy groups, halogen atoms, or 1-3 (C₁-C₅)-alkoxy groups,
an optionally substituted (C₃-C₇)-cycloalkyl group, an optionally substituted heterocyclyl group, an optionally substituted aryl group, a monocyclic or bicyclic heteroaryl group that optionally is substituted by one or more groups selected from (C₁-C₅)-alkyl groups (which optionally themselves can be substituted by 1-3 hydroxy groups or 1-3 COOR¹⁰, groups, whereby R¹⁰ means any hydroxy protective group, a benzyl group or a C₁-C₁₀-alkyl group), (C₁-C₅)-alkoxy groups, halogen atoms, or exomethylene groups and that optionally contains 1-3 nitrogen atoms and/or 1-2 oxygen atoms and/or 1-2 sulfur atoms and/or 1-2 keto groups, whereby this group can be linked via any position to the amine of the 5H-benzocycloheptene system and optionally can be hydrogenated at one or more sites,
A means a —CR⁶R⁷—CH₂— group or a —CH₂—CR⁶R⁷— group,
D means a —CR⁴R⁵—CH₂— group or a —CH₂—CR⁴R⁵— group,
R⁴ means a hydroxy group, a group OR¹⁰ or an O(CO)R¹⁰ group,
R⁵ means a (C₁-C₅)-alkyl group or an optionally partially or completely fluorinated (C₁-C₅)-alkyl group, a (C₃-C₇)cycloalkyl group, a (C₁-C₈)alkylene(C₃-C₇)cycloalkyl group, a (C₂-C₈)alkenylene(C₃-C₇)cycloalkyl group, a heterocyclyl group, a (C₁-C₈)alkyleneheterocyclyl group, a (C₂-C₈)alkenyleneheterocyclyl group, an aryl group, a (C₁-C₈)alkylenaryl group, a (C₂-C₈)alkenylenaryl group, a (C₂-C₈)alkinylenaryl group, a monocyclic or bicyclic heteroaryl group that optionally is substituted by 1-2 keto groups, 1-2 (C₁-C₅)-alkyl groups, 1-2 (C₁-C₅)-alkoxy groups, 1-3 halogen atoms, 1-2 exomethylene groups and that contains 1-3 nitrogen atoms and/or 1-2 oxygen atoms and/or 1-2 sulfur atoms; a (C₁-C₈)alkylheteroaryl group or a (C₂-C₈)alkenylheteroaryl group, whereby these groups can be linked via any position to the 5H-benzocycloheptene system and optionally can be hydrogenated at one or more sites,
R⁶ and R⁷, independently of one another, mean a hydrogen atom, a methyl or ethyl group or, together with the carbon atom of the 5H-benzocycloheptene system, a (C₃-C₆)-cycloalkyl ring.

A subject of the invention relates to stereoisomers of general formula (I), in which
R¹ and R², independently of one another, mean a hydrogen atom, a hydroxy group, a halogen atom, an optionally substituted (C₁-C₁₀)-alkyl group, a (C₁-C₁₀)-alkoxy group, a (C₁-C₁₀)-alkylthio group, a (C₁-C₅)-perfluoroalkyl group, a cyano group, or a nitro group, or
R¹ and R² together mean a group that is selected from the groups —O—(CH₂)ₙ—O—, —O—(CH₂)ₙ—CH₂—, —O—CH═CH—, —(CH₂)ₙ₊₂—, —NH—(CH₂)ₙ₊₁—, —N(C₁-C₃-alkyl)-(CH₂)ₙ₊₁—, and —NH—N═CH—,
whereby n=1 or 2, and the terminal oxygen atoms and/or carbon atoms and/or nitrogen atoms are linked to directly adjacent ring-carbon atoms, or NR⁸R⁹, whereby R⁸ and R⁹, independently of one another, can be hydrogen, C₁-C₅-alkyl or (CO)—C₁-C₅-alkyl,
R¹¹ means a hydrogen atom, a hydroxy group, a halogen atom, a cyano group, an optionally substituted (C₁-C₁₀)-alkyl group, a (C₁-C₁₀)-alkoxy group, a (C₁-C₁₀)-alkylthio group, or a (C₁-C₅)-perfluoroalkyl group,
R¹² means a hydrogen atom, a hydroxy group, a halogen atom, a cyano group, an optionally substituted (C₁-C₁₀)-alkyl group, or a (C₁-C₁₀)-alkoxy group,
R³ means a C₁-C₁₀-alkyl group, which optionally can be substituted by a group that is selected from 1-3 hydroxy groups, halogen atoms, or 1-3 (C₁-C₅)-alkoxy groups,
an optionally substituted (C₃-C₇)-cycloalkyl group, an optionally substituted heterocyclyl group, an optionally substituted aryl group, a monocyclic or bicyclic heteroaryl group that optionally is substituted by one or more groups selected from (C₁-C₅)-alkyl groups (which optionally themselves can be substituted by 1-3 hydroxy groups or 1-3 COOR¹⁰ groups, whereby R¹⁰ means any hydroxy protective group, a benzyl group or a C₁-C₁₀-alkyl group), (C₁-C₅)-alkoxy groups, halogen atoms, or exomethylene groups and that optionally contains 1-3 nitrogen atoms and/or 1-2 oxygen atoms and/or 1-2 sulfur atoms and/or 1-2 keto groups, whereby this group can be linked via any position to the amine of the 5H-benzocycloheptene system and optionally can be hydrogenated at one or more sites,
A means a —CR⁶R⁷—CH₂— group or a —CH₂—CR⁶R⁷— group,
D means a —CR⁴R⁵—CH₂— group or a —CH₂—CR⁴R⁵— group,
R⁴ means a hydroxy group, a group OR¹⁰ or an O(CO)R¹⁰ group,
R⁵ means a (C₁-C₅)-alkyl group or an optionally partially or completely fluorinated (C₁-C₅)-alkyl group, a (C₃-C₇)cycloalkyl group, a (C₁-C₈)alkylene(C₃-C₇)cycloalkyl group, a (C₂-C₈)alkenylene(C₃-C₇)cyclo-alkyl group, a heterocyclyl group, a (C₁-C₈)alkyleneheterocyclyl group, a (C₂-C₈)alkenyleneheterocyclyl group, an aryl group, a (C₁-C₈)alkylenaryl group, a (C₂-C₈)alkenylenaryl group, a (C₂-C₈)alkinylenaryl group, a monocyclic or bicyclic heteroaryl group that optionally is substituted by 1-2 keto groups, 1-2 (C₁-C₅)-alkyl groups, 1-2 (C₁-C₅)-alkoxy groups, 1-3 halogen atoms, or 1-2 exomethylene groups and that contains 1-3 nitrogen atoms and/or 1-2 oxygen atoms and/or 1-2 sulfur atoms, a (C₁-C₈)alkylheteroaryl group or a (C₂-C₈)alkenylheteroaryl group, whereby these groups can be linked via any position to the 5H-benzocycloheptene system and optionally can be hydrogenated at one or more sites,
R⁶ and R⁷, independently of one another, mean a hydrogen atom, a methyl or ethyl group or, together with the carbon atom of the 5H-benzocycloheptene system, a (C₃-C₆)-cycloalkyl ring.

Compounds of general formula I, in which
R¹ and R², independently of one another, mean a hydrogen atom, a hydroxy group, a halogen atom, a (C₁-C₁₀)-alkyl group, a (C₁-C₁₀)-alkoxy group, a (C₁-C₁₀)-alkylthio group, a (C₁-C₅)-perfluoroalkyl group, a cyano group, or a nitro group, or R¹ and R² together mean a group that is selected from the groups —O—(CH₂)ₙ—O—, —O—(CH₂)ₙ—CH₂—, —O—CH═CH—, and —(CH₂)ₙ₊₂—, whereby n=1 or 2, and the terminal atoms are linked to directly adjacent ring-carbon atoms,
or NR⁸R⁹,
whereby R⁸ and R⁹, independently of one another, can be hydrogen, C₁-C₅-alkyl or (CO)-C₁-C₅-alkyl,
R³ means a C₁-C₁₀-alkyl group, which optionally can be substituted by a group that is selected from 1-3 hydroxy groups, halogen atoms, or 1-3 (C₁-C₅)-alkoxy groups,
an optionally substituted phenyl group or a naphthyl group,
a monocyclic or bicyclic heteroaryl group that optionally is substituted by 1-2 keto groups, 1-2 (C₁-C₅)-alkyl groups, 1-2 (C₁-C₅)-alkoxy groups, 1-3 halogen atoms, or 1-2 exomethylene groups and that contains 1-3 nitrogen atoms and/or 1-2 oxygen atoms and/or 1-2 sulfur atoms, whereby these groups can be linked via any position to the amine of the benzocycloheptene system and optionally can be hydrogenated at one or more sites,
A means a —CR⁶R⁷—CH₂— group or a —CH₂—CR⁶R⁷— group,
D means a —CR⁴R⁵—CH₂— group or a —CH₂—CR⁴R⁵— group,
R⁴ means a hydroxy group,
R⁵ means a (C₁-C₅)-alkyl group or an optionally partially or completely fluorinated (C₁-C₅)-alkyl group, an aryl group, a (C₁-C₈)alkylenaryl group, a (C₂-C₈)alkenylenaryl group, a (C₃-C₇)cycloalkyl group, a (C₁-C₈)alkylene(C₃-C₇)cycloalkyl group, or a (C₂-C₈)alkenylene(C₃-C₇)cycloalkyl group,
R⁶ and R⁷, independently of one another, mean a hydrogen atom, a methyl or ethyl group, or, together with the carbon atom of the benzocycloheptene system, a (C₃-C₆)-cycloalkyl ring,
   are a sub-group of the compounds according to the invention.

Stereoisomers of general formula (I), in which
R¹ and R², independently of one another, mean a hydrogen atom, a hydroxy group, a halogen atom, a (C₁-C₁₀)-alkyl group, a (C₁-C₁₀)-alkoxy group, a (C₁-C₁₀)-alkylthio group, a (C₁-C₅)-perfluoroalkyl group, a cyano group, or a nitro group, or R¹ and R² together mean a group that is selected from the groups —O—(CH₂)ₙ—O—, —O—(CH₂)ₙ—CH₂—, —O—CH═CH—, or —(CH₂)ₙ₊₂—, whereby n=1 or 2, and the terminal atoms are linked to directly adjacent ring-carbon atoms, or NR⁸R⁹,
whereby R⁸ and R⁹, independently of one another, can be hydrogen, C₁-C₅-alkyl or (CO)—C₁-C₅-alkyl,
R³ means a C₁-C₁₀-alkyl group, which optionally can be substituted by a group that is selected from 1-3 hydroxy groups, halogen atoms, or 1-3 (C₁-C₅)-alkoxy groups,
an optionally substituted phenyl group,
a monocyclic or bicyclic heteroaryl group that optionally is substituted by 1-2 keto groups, 1-2 (C₁-C₅)-alkyl groups, 1-2 (C₁-C₅)-alkoxy groups, 1-3 halogen atoms, or 1-2 exomethylene groups and that contains 1-3 nitrogen atoms and/or 1-2 oxygen atoms and/or 1-2 sulfur atoms, whereby these groups can be linked via any position to the amine of the benzocycloheptene system and optionally can be hydrogenated at one or more sites,
A means a —CR⁶R⁷—CH₂— group or a —CH₂—CR⁶R⁷— group,
D means a —CR⁴R⁵—CH₂— group or a —CH₂—CR⁴R⁵— group,
R⁴ means a hydroxy group,
R⁵ means a (C₁-C₅)-alkyl group or an optionally partially or completely fluorinated (C₁-C₅)-alkyl group, an aryl group, a (C₁-C₈)alkylenaryl group, a (C₂-C₈)alkenylenaryl group, a (C₃-C₇)cycloalkyl group, a (C₁-C₈)alkylene(C₃-C₇)cycloalkyl group, or a (C₂-C₈)alkenylene(C₃-C₇)cycloalkyl group,
R⁶ and R⁷, independently of one another, mean a hydrogen atom, a methyl or ethyl group, or, together with the carbon atom of the 5H-benzocycloheptene system, a (C₃-C₆)-cycloalkyl ring,
   are preferred.

Compounds of general formula I, in which
R¹ and R², independently of one another, mean a hydrogen atom, a hydroxy group, a halogen atom, a (C₁-C₅)-alkyl group, a (C₁-C₅)-alkoxy group, a (C₁-C₅)-perfluoroalkyl group, or a cyano group, or R¹ and R² together mean a group that is selected from the groups —O—(CH₂)ₙ—O—, —O—(CH₂)ₙ—CH₂—, —O—CH═CH—, and —(CH₂)ₙ₊₂—,
whereby n=1 or 2, and the terminal atoms are linked to directly adjacent ring-carbon atoms,
R³ means a C₁-C₁₀-alkyl group, which optionally can be substituted by 1-3 hydroxy groups, halogen atoms; a phenyl, phthalidyl, isoindolyl, dihydroindolyl, dihydroisoindolyl, dihydroisoquinolinyl, thiophthalidyl, benzoxazinonyl, phthalazinonyl, quinolinyl, isoquinolinyl, quinolonyl, isoquinolonyl, indazolyl, benzothiazolyl, quinazolinyl, quinoxalinyl, cinnolinyl, phthalazinyl, 1,7- or 1,8-naphthyridinyl, dihydroindolonyl, dihydroisoindolonyl, benzimidazolyl or indolyl group that optionally is substituted with C₁-C₅-alkyl, halogen, hydroxy, or C₁-C₅-alkoxy, whereby these groups can be linked via any position to the amine of the 5H-benzocycloheptene system and optionally can be substituted in one or more places with 1-2 keto groups, 1-2 (C₁-C₃)-alkyl groups, 1-2 (C₁-C₃)-alkoxy groups, 1-3 halogen atoms, or 1-2 exomethylene groups, and optionally can be hydrogenated at one or more sites,
A means a —CR⁶R⁷—CH₂— group or a —CH₂—CR⁶R⁷— group,
D means a —CR⁴R⁵—CH₂— group or a —CH₂—CR⁴R⁵— group,
R⁴ means a hydroxy group,
R⁵ means a (C₁-C₅)-alkyl group or an optionally partially or completely fluorinated (C₁-C₅)-alkyl group,
R⁶ and R⁷, independently of one another, mean a hydrogen atom, a methyl group or ethyl group, or, together with the carbon atom of the benzocycloheptene system, a (C₃-C₆)-cycloalkyl ring,
   represent a special sub-group.

Stereoisomers of general formula I, in which
R¹ and R², independently of one another, mean a hydrogen atom, a hydroxy group, a halogen atom, a (C₁-C₅)-alkyl group, a (C₁-C₅)-alkoxy group, a (C₁-C₅)-perfluoroalkyl group, or a cyano group, or R¹ and R² together mean a group that is selected from the groups —O—(CH₂)ₙ—O—, —O—(CH₂)ₙ—CH₂—, —O—CH═CH—, and —(CH₂)ₙ₊₂—,
whereby n=1 or 2, and the terminal oxygen atoms and/or carbon atoms are linked to directly adjacent ring-carbon atoms,
R³ means a C₁-C₁₀-alkyl group, which optionally can be substituted by 1-3 hydroxy groups, halogen atoms; a phenyl, phthalidyl, isoindolyl, dihydroindolyl, dihydroisoindolyl, dihydroisoquinolinyl, thiophthalidyl, benzoxazinonyl, phthalazinonyl, quinolinyl, isoquinolinyl, quinolonyl, isoquinolonyl, indazolyl, benzothiazolyl, quinazolinyl, quinoxalinyl, cinnolinyl, phthalazinyl, 1,7- or 1,8-naphthyridinyl, dihydroindolonyl, dihydroisoindolonyl, benzimidazolyl or indolyl group that optionally is substituted with C₁-C₅-alkyl, halogen, hydroxy, or C₁-C₅-alkoxy, whereby these groups can be linked via any position to the amine of the 5H-benzocycloheptene system and optionally can be substituted in one or more places with 1-2 keto groups, 1-2 (C₁-C₃)-alkyl groups, 1-2 (C₁-C₃)-alkoxy groups, 1-3 halogen atoms, or 1-2 exomethylene groups, and optionally can be hydrogenated at one or more sites,
A means a —CR⁶R⁷—CH₂— group or a —CH₂—CR⁶R⁷— group,
D means a —CR⁴R⁵—CH₂— group or a —CH₂—CR⁴R⁵— group,
R⁴ means a hydroxy group,
R⁵ means a (C₁-C₅)-alkyl group or an optionally partially or completely fluorinated (C₁-C₅)-alkyl group,
R⁶ and R⁷, independently of one another, mean a hydrogen atom, a methyl group or ethyl group, or, together with the carbon atom of the benzocycloheptene system, a (C₃-C₆)-cycloalkyl ring,
   are another subject of the invention.

Stereoisomers of general formula (I), in which
R¹ and R², independently of one another, mean a hydrogen atom, a hydroxy group, a halogen atom, a (C₁-C₅)-alkyl group, a (C₁-C₅)-perfluoroalkyl group, a cyano group, a (C₁-C₅)-alkoxy group, or together a (C₁-C₂)-alkylenedioxy group, whereby then R¹ and R²must be directly adjacent,
R³ means a phenyl, phthalidyl, isoindolyl, dihydroindolyl, dihydroisoindolyl, dihydroisoquinolinyl, thiophthalidyl, benzoxazinonyl, phthalazinonyl, quinolinyl, isoquinolinyl, quinolonyl, isoquinolonyl, indazolyl, benzothiazolyl, quinazolinyl, quinoxalinyl, cinnolinyl, phthalazinyl, 1,7- or 1,8-naphthyridinyl, dihydroindolonyl, dihydroisoindolonyl, benzimidazolyl or indolyl group that optionally is substituted with C₁-C₅-alkyl, halogen, hydroxy, or C₁-C₅-alkoxy,
whereby these groups can be linked via any position to the amine of the benzocycloheptene system and optionally can be substituted in one or more places with 1-2 keto groups, 1-2 (C₁-C₃)-alkyl groups, or 1-2 exomethylene groups and optionally can be hydrogenated at one or more sites,
A means a —CR⁶R⁷—CH₂— group or a —CH₂—CR⁶R⁷— group,
D means a —CR⁴R⁵—CH₂— group or a —CH₂—CR⁴R⁵— group,
R⁴ means a hydroxy group,
R⁵ means a (C₁-C₅)-alkyl group or an optionally partially or completely fluorinated (C₁-C₅)-alkyl group,
R⁶ and R⁷, independently of one another, mean a hydrogen atom, a methyl or ethyl group or, together with the carbon atom of the benzocycloheptene system, a (C₃-C₆)-cycloalkyl ring,
   are especially preferred.

Compounds of general formula I, in which
R¹ and R², independently of one another, mean a hydrogen atom, a hydroxy group, a halogen atom, a (C₁-C₁₀)-alkyl group, a (C₁-C₁₀)-alkoxy group, a (C₁-C₁₀)-alkylthio group, a (C₁-C₅)-perfluoroalkyl group, a cyano group, or a nitro group, or R¹ and R² together mean a group that is selected from the groups —O—(CH₂)ₙ—O—, —O—(CH₂)ₙ—CH₂—, —O—CH═CH—, or —(CH₂)ₙ₊₂—, whereby n=1 or 2, and the terminal atoms are linked to directly adjacent ring-carbon atoms,
or N⁸R⁹,
whereby R⁸ and R⁹ independently of one another, can be hydrogen, C₁-C₅-alkyl or (CO)-C₁-C₅-alkyl,
R¹¹ and R¹² mean a hydrogen atom,
R³ means a C₁-C₁₀-alkyl group, which optionally can be substituted by a group that is selected from 1-3 hydroxy groups, halogen atoms, or 1-3 (C₁-C₅)-alkoxy groups,
an optionally substituted phenyl group or a naphthyl group,
a monocyclic or bicyclic heteroaryl group that optionally is substituted by 1-2 keto groups, 1-2 (C₁-C₅)-alkyl groups, 1-2 (C₁-C₅)-alkoxy groups, 1-3 halogen atoms, or 1-2 exomethylene groups and that contains 1-3 nitrogen atoms and/or 1-2 oxygen atoms and/or 1-2 sulfur atoms, whereby these groups can be linked via any position to the amine of the benzocycloheptene system and optionally can be hydrogenated at one or more sites,
A means a —CR⁶R⁷—CH₂— group or a —CH₂—CR⁶R⁷— group,
D means a —CR⁴R⁵—CH₂— group or a —CH₂—CR⁴R⁵— group,
R⁴ means a hydroxy group,
R⁵ means a (C₁-C₅)-alkyl group or an optionally partially or completely fluorinated (C₁-C₅)-alkyl group, an aryl group, a (C₁-C₈)alkylenaryl group, a (C₂-C₈)alkenylenaryl group, a (C₃-C₇)cycloalkyl group, a (C₁-C₈)alkylene(C₃-C₇)cycloalkyl group, or a (C₂-C₈)alkenylene(C₃-C₇)cycloalkyl group,
R⁶ and R⁷, independently of one another, mean a hydrogen atom, a methyl or ethyl group, or, together with the carbon atom of the benzocycloheptene system, a (C₃-C₆)-cycloalkylring,
   are a subject of the invention.

Stereoisomers of general formula (I), in which

R¹ and R², independently of one another, mean a hydrogen atom, a hydroxy group, a halogen atom, a (C₁-C₁₀)-alkyl group, a (C₁-C₁₀)-alkoxy group, a (C₁-C₁₀)-alkylthio group, a (C₁-C₅)-perfluoroalkyl group, a cyano group, or a nitro group, or R¹ and R² together mean a group that is selected from the groups —O—(CH₂)ₙ—O—, —O—(CH₂)ₙ—CH₂—, —O—CH═CH—, and —(CH₂)ₙ₊₂—, whereby n=1 or 2, and the terminal atoms are linked to directly adjacent ring-carbon atoms, or NR⁸R⁹,
whereby R⁸ and R⁹, independently of one another, can be hydrogen, C₁-C₅-alkyl or (CO)—C₁-C₅-alkyl,
R¹¹ and R¹² mean a hydrogen atom,
R³ means a C₁-C₁₀-alkyl group, which optionally can be substituted by a group that is selected from 1-3 hydroxy groups, halogen atoms, or 1-3 (C₁-C₅)-alkoxy groups,
an optionally substituted phenyl group,
a monocyclic or bicyclic heteroaryl group that optionally is substituted by 1-2 keto groups, 1-2 (C₁-C₅)-alkyl groups, 1-2 (C₁-C₅)-alkoxy groups, 1-3 halogen atoms, or 1-2 exomethylene groups and that contains 1-3 nitrogen atoms and/or 1-2 oxygen atoms and/or 1-2 sulfur atoms, whereby these groups can be linked via any position to the amine of the benzocycloheptene system and optionally can be hydrogenated at one or more sites,
A means a —CR⁶R⁷—CH₂— group or a —CH₂—CR⁶R⁷— group,
D means a —CR⁴R⁵—CH₂— group or a —CH₂—CR⁴R⁵— group,
R⁴ means a hydroxy group,
R⁵ means a (C₁-C₅)-alkyl group or an optionally partially or completely fluorinated (C₁-C₅)-alkyl group, an aryl group, a (C₁-C₈)alkylenaryl group, a (C₂-C₈)alkenylene aryl group, a (C₃-C₇)cycloalkyl group, a (C₁-C₈)alkylene(C₃-C₇)cycloalkyl group, or a (C₂-C₈)alkyenylene(C₃-C₇)cycloalkyl group,
R⁶ and R⁷, independently of one another, mean a hydrogen atom, a methyl group or ethyl group, or, together with the carbon atom of the
benzocycloheptene system, a (C₃-C₆)-cycloalkyl ring,
   are preferred.

Stereoisomers of general formula I, in which
R¹ and R², independently of one another, mean a hydrogen atom, a hydroxy group, a halogen atom, a (C₁-C₅)-alkyl group, a (C₁-C₅)-alkoxy group, a (C₁-C₅)-perfluoroalkyl group, or a cyano group, or R¹ and R² together mean a group that is selected from the groups —O—(CH₂)ₙ—O—, —O—(CH₂)ₙ—CH₂—, —O—CH═CH—, or —(CH₂)ₙ₊₂—.
whereby n=1 or 2, and the terminal atoms are linked to directly adjacent ring-carbon atoms,
R¹¹ and R¹² mean a hydrogen atom,
R³ means a C₁-C₁₀-alkyl group, which optionally can be substituted by 1-3 hydroxy groups, halogen atoms; a phenyl, phthalidyl, isoindolyl, dihydroindolyl, dihydroisoindolyl, dihydroisoquinolinyl, thiophthalidyl, benzoxazinonyl, phthalazinonyl, quinolinyl, isoquinolinyl, quinolonyl, isoquinolonyl, indazolyl,-benzothiazolyl, quinazolinyl, quinoxalinyl, cinnolinyl, phthalazinyl, 1,7- or 1,8-naphthyridinyl, dihydroindolonyl, dihydroisoindolonyl, benzimidazolyl or indolyl group that optionally is substituted with C₁-C₅-alkyl, halogen, hydroxy, or C₁-C₅-alkoxy, whereby these groups can be linked via any position to the amine of the 5H-benzocycloheptene system and optionally can be substituted in one or more places with 1-2 keto groups, 1-2 (C₁-C₃)-alkyl groups, 1-2 (C₁-C₃)-alkoxy groups, 1-3 halogen atoms, or 1-2 exomethylene groups, and optionally can be hydrogenated at one or more sites,
A means a —CR⁶R⁷—CH₂— group or a —CH₂—CR⁶R⁷— group,
D means a —CR⁴R⁵—CH₂— group or a —CH₂—CR⁴R⁵— group,
R⁴ means a hydroxy group,
R⁵ means a (C₁-C₅)-alkyl group or an optionally partially or completely fluorinated (C₁-C₅)-alkyl group,
R⁶ and R⁷, independently of one another, mean a hydrogen atom, a methyl group or ethyl group, or, together with the carbon atom of the benzocycloheptene system, a (C₃-C₆)-cycloalkyl ring,
   are a special subject of the invention.

Stereoisomers of general formula (I), in which
R¹ and R², independently of one another, mean a hydrogen atom, a hydroxy group, a halogen atom, a (C₁-C₅)-alkyl group, a (C₁-C₅)-perfluoroalkyl group, a cyano group, or a (C₁-C₅)-alkoxy group, or R¹ and R² together mean a group that is selected from the groups —O—(CH₂)ₙ—O—, —O—(CH₂)ₙ—CH₂—, —O—CH═CH—, and —(CH₂)ₙ₊₂—,
whereby n=1 or 2, and the terminal atoms are linked to directly adjacent ring-carbon atoms,
R¹¹ and R¹² mean a hydrogen atom,
R³ means a phenyl, phthalidyl, isoindolyl, dihydroindolyl, dihydroisoindolyl, dihydroisoquinolinyl, thiophthalidyl, benzoxazinonyl, phthalazinonyl, quinolinyl, isoquinolinyl, quinolonyl, isoquinolonyl, indazolyl, benzothiazolyl, quinazolinyl, quinoxalinyl, cinnolinyl, phthalazinyl, 1,7- or 1,8-naphthyridinyl, dihydroindolonyl, dihydroisoindolonyl, benzimidazolyl or indolyl group that optionally is substituted with C₁-C₅-alkyl, halogen, hydroxy, or C₁-C₅-alkoxy,
whereby these groups can be linked via any position to the amine of the benzocycloheptene system and optionally can be substituted in one or more places with 1-2 keto groups, 1-2 (C₁-C₃)-alkyl groups, 1-2 exomethylene groups, and optionally can be hydrogenated at one or more sites,
A means a —CR⁶R⁷—CH₂— group or a —CH₂—CR⁶R⁷— group,
D means a —CR⁴R⁵—CH₂— group or a —CH₂—CR⁶R⁵— group,
R⁴ means a hydroxy group,
R⁵ means a (C₁-C₅)-alkyl group or an optionally partially or completely fluorinated (C₁-C₅)-alkyl group,
R⁶ and R⁷, independently of one another, mean a hydrogen atom, a methyl group or ethyl group, or, together with the carbon atom of the
benzocycloheptene system, a (C₃-C₆)-cycloalkyl ring,
   are preferred.

Stereoisomers of general formula (I), in which
R¹ and R², independently of one another, mean a hydrogen atom, a hydroxy group, a halogen atom, a (C₁-C₅)-alkyl group, a (C₁-C₅)-perfluoroalkyl group, a cyano group, a (C₁-C₅)-alkoxy group, or, together, a (C₁-C₂)-alkylenedioxy group, whereby then R¹ and R² must be directly adjacent,
R¹¹ and R¹² mean a hydrogen atom,
R³ means a phenyl, furanyl, thienyl, pyrazolyl, pyrrolyl, oxazolyl, thiazolyl, imidazolyl, isoxazolyl, isothiazolyl, oxadiazolyl, triazolyl, tetrazolyl, thiadiazolyl, pyridyl, pyrimidinyl, pyridazinyl, pyrazinyl, triazinyl, azaindolizinyl, phthalidyl, thiophthalidyl, indolyl, isoindolyl, dihydroindolyl, dihydroisoindolyl, indazolyl, benzothiazolyl, indolonyl, dihydroindolonyl, isoindolonyl, dihydroisoindolonyl, benzofuranyl, benzimidazolyl, indolizinyl, isobenzofuranyl, azaindolyl, azaisoindolyl, furanopyridyl, furanopyrimidinyl, furanopyrazinyl, furanopyridazinyl, dihydrobenzofuranyl, dihydrofuranopyridyl, dihydrofuranopyrimidinyl, dihydrofuranopyrazinyl, dihydrofuranopyridazinyl, dihydrobenzofuranyl, coumarinyl, isocoumarinyl, dihydroisoquinolinyl, dihydroquinolinyl, benzoxazinonyl, phthalazinonyl, quinolinyl, isoquinolinyl, quinolonyl, isoquinolonyl, quinazolinyl, quinoxalinyl, cinnolinyl, phthalazinyl, 1,7- or 1,8-naphthyridinyl group, tetrahydropyranyl, 2H-pyranyl, 4H-pyranyl, piperidyl, tetrahydropyridyl, dihydropyridyl, 1H-pyridin-2-onyl, 1H-pyridin-4-onyl, 4-aminopyridyl, 1H-pyridin-4-ylidenaminyl, chromanyl, isochromanyl, chromenyl, isochromenyl, thiochromanyl, decahydroquinolinyl, tetrahydroquinolinyl, dihydroquinolinyl, 5,6,7,8-tetrahydro-1H-quinolin-4-onyl, decahydroisoquinolinyl, tetrahydroisoquinolinyl, dihydroisoquinolinyl, 3,4-dihydro-2H-benz[1,4]oxazinyl, 1,2-dihydro[1,3]benzoxazin-4-onyl, 3,4-dihydrobenz[1,4]oxazin-4-onyl, 3,4-dihydro-2H-benzo[1,4]thiazinyl, 4H-benzo[1,4]thiazinyl, 1,2,3,4-tetrahydroquinoxalinyl, 1H-cinnolin-4-onyl, 3H-quinazolin-4-onyl, 1H-quinazolin-4-onyl, 3,4-dihydro-1H-quinoxalin-2-onyl, 2,3-1,2,3,4-tetrahydro[1,5]naphthyridinyl, dihydro-1H-[1,5]naphthyridyl, 1H-[1,5]naphthyrid-4-onyl, 5,6,7,8-tetrahydro-1H-naphthyridin-4-onyl, 1,2-dihydropyrido[3,2-d][1,3]oxazin-4-onyl, octahydro-1H-indolyl, 2,3-dihydro-1H-indolyl, octahydro-2H-isoindolyl, 1,3-dihydro-2H-isoindolyl, 1,2-dihydroindazolyl, 1H-pyrrolo[2,3-b]pyridyl, 2,3-dihydro-1H-pyrrolo[2,3-b]pyridyl, or 2,2-dihydro-1H-pyrrolo[2,3-b]pyridin-3-onyl group, whereby these groups can be linked via any position to the amine of the benzocycloheptene system and optionally can be substituted in one or more places with 1-2 keto groups, 1-2 (C₁-C₃)-alkyl groups, or 1-2 exomethylene groups and optionally can be hydrogenated at one or more sites,
A means a —CR⁶R⁷—CH₂— group or a —CH₂—CR⁶R⁷— group,
D means a —CR⁴R⁵—CH₂— group or a —CH₂—CR⁴R⁵— group,
R⁴ means a hydroxy group,
R⁵ means a (C₁-C₅)-alkyl group or an optionally partially or completely fluorinated (C₁-C₅)-alkyl group,
R⁶ and R⁷, independently of one another, mean a hydrogen atom, a methyl or ethyl group or, together with the carbon atom of the benzocycloheptene system, a (C₃-C₆)-cycloalkyl ring,
   are especially preferred.

Stereoisomers of general formula (I), in which
R¹ and R², independently of one another, mean a hydrogen atom, a hydroxy group, a halogen atom, a (C₁-C₅)-alkyl group, a (C₁-C₅)-perfluoroalkyl group, a cyano group, a (C₁-C₅)-alkoxy group, or together a (C₁-C₂)-alkenylenedioxy group, whereby then R¹ and R² must be directly adjacent,
R¹¹ and R¹² mean a hydrogen atom,
R³ means a phenyl, phthalidyl, isoindolyl, dihydroindolyl, dihydroisoindolyl, dihydroisoquinolinyl, thiophthalidyl, benzoxazinonyl, phthalazinonyl, quinolinyl, isoquinolinyl, quinolonyl, isoquinolonyl, indazolyl, benzothiazolyl, quinazolinyl, quinoxalinyl, cinnolinyl, phthalazinyl, 1,7- or 1,8-naphthyridinyl, dihydroindolonyl, dihydroisoindolonyl, benzimidazolyl or indolyl group that optionally is substituted with C₁-C₅-alkyl, halogen, hydroxy, or C₁-C₅-alkoxy,
whereby these groups can be linked via any position to the amine of the benzocycloheptene system and optionally can be substituted in one or more places with 1-2 keto groups, 1-2 (C₁-C₃)-alkyl groups, or 1-2 exomethylene groups, and optionally can be hydrogenated at one or more sites,
A means a —CR⁶R⁷—CH₂— group or a —CH₂—CR⁶R⁷— group,
D means a —CR⁴R⁵—CH₂— group or a —CH₂—CR⁴R⁵— group,
R⁴ means a hydroxy group,
R⁵ means a (C₁-C₅)-alkyl group or an optionally partially or completely fluorinated (C₁-C₅)-alkyl group,
R⁶ and R⁷ independently of one another, mean a hydrogen atom, a methyl or ethyl group, or, together with the carbon atom of the benzocycloheptene system, a (C₃-C₆)-cycloalkylring,
   are especially preferred.

Stereoisomers of general formula (I), in which
R¹ and R², independently of one another, mean a hydrogen atom, a hydroxy group, a halogen atom, a (C₁-C₃)-alkyl group, a (C₁-C₃)-perfluoroalkyl group, a cyano group, or a (C₁-C₃)-alkoxy group,
R¹¹ and R¹² mean a hydrogen atom,
R³ means a phenyl, phthalidyl, isoindolyl, dihydroindolyl, dihydroisoindolyl, dihydroisoquinolinyl, thiophthalidyl, benzoxazinonyl, phthalazinonyl, quinolinyl, isoquinolinyl, quinolonyl, isoquinolonyl, indazolyl, benzothiazolyl, quinazolinyl, quinoxalinyl, cinnolinyl, phthalazinyl, 1,7- or 1,8-naphthyridinyl, dihydroindolonyl, dihydroisoindolonyl, benzimidazolyl or indolyl group that optionally is substituted with C₁-C₃-alkyl, halogen, hydroxy, or C₁-C₃-alkoxy,
whereby these groups can be linked via any position to the amine of the benzocycloheptene system and optionally can be substituted in one or more places with 1-2 keto groups, 1-2 (C₄-C₃)-alkyl groups, or 1-2 exomethylene groups and optionally can be hydrogenated at one or more sites,
A means a —CR⁶R⁷—CH₂— group or a —CH₂—CR⁶R⁷— group,
D means a —CR⁴R⁵—CH₂— group or a —CH₂—CR⁴R⁵— group,
R⁴ means a hydroxy group,.
R⁵ means a completely fluorinated (C₁-C₃)-alkyl group,
R⁶ and R⁷, independently of one another, mean a hydrogen atom, a methyl or ethyl group or, together with the carbon atom of the benzocycloheptene system, a (C₃-C₆)-cycloalkyl ring,
   are especially preferred.

Stereoisomers of general formula (I), in which
R¹ and R², independently of one another, mean a hydrogen atom, a hydroxy group, a halogen atom, a (C₁-C₃)-alkyl group, a CF₃ group, a cyano group, or a methoxy group,
R¹¹ and R¹² mean a hydrogen atom,
R³ means an isoindolyl, dihydroindolyl, dihydroisoindolyl, dihydroisoquinolinyl, quinolinyl, isoquinolinyl, quinazolinyl, quinolonyl, isoquinolonyl, indazolyl, dihydroindolonyl or dihydroisoindolonyl group that optionally is substituted with C₁-C₃-alkyl, halogen, hydroxy, C₁-C₃-alkoxy or methylpyrrolidin-on-5-yl, whereby these groups can be linked via any position to the amine of the benzocycloheptene system and optionally can be substituted in one or more places with 1-2 keto groups, 1-2 (C₁-C₃)-alkyl groups, or 1-2 exomethylene groups, and optionally can be hydrogenated at one or more sites,
A means a —CR⁶R⁷—CH₂— group or a —CH₂—CR⁶R⁷— group,
D means a —CR⁴R⁵—CH₂— group or a —CH₂—CR⁴R⁵— group,
R⁴ means a hydroxy group,
R⁵ means a completely fluorinated (C₁-C₃)-alkyl group,
R⁶ and R⁷, independently of one another, mean a hydrogen atom, a methyl group or ethyl group,
   are quite especially preferred.

Compounds that carry one or two substituents—selected from the group C₁-C₅-alkyl, C₁-C₅-alkoxy, C₁-C₅-perfluoroalkyl, halogen, hydroxy, nitro, —O—(CH₂)ₙ—O—, —O—(CH₂)ₙ—CH₂—, —O—CH═CH—, —(CH₂)ₙ₊₂—, —NH—(CH₂)ₙ₊₁, N(C₁-C₃-alkyl)-(CH₂)ₙ₊₁, or —NH—N═CH—, whereby n=1 or 2, and the terminal atoms of the divalent substituents are linked to directly adjacent ring-carbon atoms—on the aromatic ring of the 5H-benzocycloheptene system are a special subject of the invention.

Compounds of general formula I according to one of claims, in which R¹ and R², independently of one another, mean a hydrogen atom, a halogen atom or a cyano group, and R¹¹ and R¹² mean a hydrogen atom, are another preferred subject.

Stereoisomers of general formula I, in which
R¹ and R², independently of one another, mean a hydrogen atom, a halogen atom or a cyano group,
R¹¹ and R¹² mean a hydrogen atom,
R³ means an indazolyl, dihydroindolonyl or quinolonyl group, which optionally can be substituted by a halogen atom or by methylpyrrolidin-2-on-5-yl,
A means —CH₂—CR⁶R⁷ or CR⁶R⁷—CH₂—,
D means —CH₂—CR⁴(R⁵),
R⁴ means a hydroxy group,
R⁵ means a C₁-C₃-perfluoroalkyl group,
R⁶, R⁷ mean a methyl or ethyl group,
   are a quite especially preferred subject of this invention.

Compounds of general formula I, in which
R¹ and R², independently of one another, mean a hydrogen atom, a bromine or chlorine atom, or a cyano group,
R¹¹ and R¹² mean a hydrogen atom,
R³ means an indazolyl, dihydroindolonyl or quinolonyl group, which optionally can be substituted by a bromine atom or by methylpyrrolidin-2-on-5-yl,
A means —CH₂—C(CH₃)₂, C(CH₃)₂—CH₂—,
D means —CH₂—C(CF₃)(OH),
R⁴ means a hydroxy group,
R⁵ means a CF₃ group,
R⁶, R⁷ mean a methyl group,
   are especially preferred.

The compounds 1-Bromo-6,7,8,9-tetrahydro-5-[(1H-indazol-4-yl)amino]-8,8-dimethyl-6-(trifluoromethyl)-5H-benzocyclohepten-6-ol,

4-{[1-Bromo-6,7,8,9-tetrahydro-6-hydroxy-8,8-dimethyl-6-(trifluoromethyl)-5H-benzocyclohepten-5-yl]amino}-1,3-dihydro-2H-indol-2-one and 4-{[1-bromo-6,7,8,9-tetrahydro-6-hydroxy-8,8-dimethyl-6-(trifluoromethyl)-5H-benzocyclohepten-5-yl]amino}-7-bromo-1,3-dihydro-2H-indol-2-one,

2-Chloro-6,7,8,9-tetrahydro-5-(1H-indazol-4-ylamino)-8,8-dimethyl-6-(trifluoromethyl)-5H-benzocyclohepten-6-ol,

4-{[2-Chloro-6,7,8,9-tetrahydro-6-hydroxy-8,8-dimethyl-6-(trifluoromethyl)-5H-benzocyclohepten-5-yl]amino}-1,3-dihydro-2H-indol-2-one,

6,7,8,9-Tetrahydro-6-hydroxy-8,8-dimethyl-5-{[7-(1-methyl-5-oxopyrrolidin-2-yl)-1H-indazol-4-yl]amino}-6-(trifluoromethyl)-5H-benzocycloheptene-2-carbonitrile,

(5R*-cis)-5-{[6,7,8,9-Tetrahydro-6-hydroxy-9,9-dimethyl-6-(trifluoromethyl)-5H-benzocyclohepten-5-yl]amino}quinolin-2-(1H)-one,

in particular (5R*-cis)-5-{[6,7,8,9-Tetrahydro-6-hydroxy-9,9-dimethyl-6-(trifluoromethyl)-5H-benzocyclohepten-5-yl]amino}quinolin-2(1H)-one are especially preferred.

The compounds in which R¹ and R² together mean the radicals —O—(CH₂)ₙ—O—, —O—(CH₂)ₙ—CH₂—, —O—CH═CH—, —(CH₂)ₙ₊₂—, —NH—(CH₂)ₙ₊₁, N(C₁-C₃-alkyl)-(CH₂)ₙ₊₁, and —NH—N═CH— are a sub-group of these compounds.

The compounds in which the alkyl radicals R¹ and R² have the meaning —(CH₂)ₙ₊₂— and thus, together with the carbon atom of the chain, form a 5- to 6-membered ring represent another sub-group.

Compounds of general formula I according to claims 1-3, in which R³ means a C₁-C₁₀-alkyl group, which optionally can be substituted by 1-3 hydroxy groups, halogen atoms, 1-3 (C₁-C₅)-alkoxy groups, an optionally substituted (C₃-C₇)-cycloalkyl group, an optionally substituted heterocyclyl group; a monocyclic or bicyclic heteroaryl group that optionally is substituted by one or more groups selected from (C₁-C₅)-alkyl groups, (C₁-C₅)-alkoxy groups, halogen atoms, or exomethylene groups and that optionally contains 1-3 nitrogen atoms and/or 1-2 oxygen atoms and/or 1-2 sulfur atoms and/or 1-2 keto groups, whereby these groups can be linked via any position to the amine of the 5H-benzocycloheptene system and optionally can be hydrogenated at one or more sites, are another subject of the invention.

Compounds of formula I, in which R³ means a C₁-C₁₀-alkyl group, which optionally can be substituted by 1-3 hydroxy groups, halogen atoms, an optionally substituted phenyl group; a monocyclic or bicyclic heteroaryl group that optionally is substituted by 1-2 keto groups, 1-2 (C₁-C₅)-alkyl groups, 1-2 (C₁-C₅)-alkoxy groups, 1-3 halogen atoms, or 1-2 exomethylene groups and that contains 1-3 nitrogen atoms and/or 1-2 oxygen atoms and/or 1-2 sulfur atoms, whereby these groups can be linked via any position to the nitrogen atom and optionally can be hydrogenated at one or more sites, are another subject of the invention.

Compounds of general formula I, in which R³ means an optionally substituted phenyl or naphthyl group, a phthalidyl, thiophthalidyl, benzoxazinonyl, phthalazinonyl, quinolinyl, isoquinolinyl, quinolonyl, isoquinolonyl, indazolyl, benzothiazolyl, quinazolinyl, quinoxalinyl, cinnolinyl, phthalazinyl, 1,7- or 1,8-naphthyridinyl, dihydroindolonyl, dihydroisoindolonyl, benzimidazolyl or indolyl group, are another subject.

Compounds of general formula I, in which R³ means a C₁-C₁₀-alkyl group, which optionally can be substituted by 1-3 hydroxy groups, halogen atoms, an optionally substituted phenyl group, phthalidyl, thiophthalidyl, benzoxazinonyl, phthalazinonyl, quinolinyl, isoquinolinyl, quinolonyl, isoquinolonyl, indazolyl, benzothiazolyl, quinazolinyl, quinoxalinyl, cinnolinyl, phthalazinyl, 1,7- or 1,8-naphthyridinyl, dihydoindolonyl, dihydroisoindolonyl, benzimidazolyl or indolyl group, are a preferred subject of the invention.

Stereoisomers of general formula I according to one of the claims, in which R³ means an indolyl, isoindolyl, dihydroindolyl, dihydroisoindolyl, quinolinyl, isoquinolinyl, dihydroquinolinyl, dihydroisoquinolinyl, quinolonyl, isoquinolonyl, dihydroquinolonyl, dihydroisoquinolonyl, indazolyl, indolonyl, isoindolonyl, dihydroindolonyl, or dihydroisoindolonyl group, are another preferred subject of the invention.

Stereoisomers of general formula I according to one of the claims, in which R³ means an indazolyl, dihydroindolonyl, dihydroisoindolonyl, quinolonyl or an isoquinolonyl group, are especially preferred.

Compounds of general formula I according to one of the claims, in which R³ means indazolyl, dihydroindolonyl and quinolonyl, which optionally can be substituted by a halogen atom or methylpyrrolidone, are a quite especially preferred subject of the invention.

The group A according to one of the claims can mean —CH₂CR⁶R⁷ or —CR⁶R⁷—CH₂—, preferably —CH₂—C(CH₃)₂ or —C(CH₃)₂—CH₂—, especially preferably —CR⁶R⁷—CH₂, and quite especially preferably —C(CH₃)₂—CH₂—.

The group D can mean a —CR⁴R⁵—CH₂— group or a —CH₂—CR⁴R⁵— group; a —CH₂—CR⁴(R⁵) group is preferred, and —CH₂—C(CF₃)(OH) is especially preferred.

The group -A-D- can mean preferably —CH₂—CR⁶R⁷—CH₂—CR⁴(R⁵)—, —CR⁶R⁷—CH₂—CH₂—CR⁴(R⁵)—, and especially preferably —CH₂—C(CH₃)₂—CH₂—C(CF₃)(OH)— or —C(CH₃)₂—CH₂—CH₂—C(CF₃)(OH)—.

The hydroxy group in R⁴ can be present in protected form, specifically preferably present by one of the common hydroxy protective groups or as a C₁-C₅ ether or ester (CO)R¹⁰.

The hydroxy group is preferred as radical R⁴.

Compounds of general formula I, in which R⁵ means a (C₁-C₅)-alkyl group or an optionally partially or completely fluorinated (C₁-C₅)-alkyl group, a (C₃-C₇)cycloalkyl group, a (C₁-C₈)alkylene(C₃-C₇)cycloalkyl group, a (C₂-C₈)-alkenylene(C₃-C₇)cycloalkyl group, a heterocyclyl group, a (C₁-C₈)alkyleneheterocyclyl group, a (C₂-C₈)alkenyleneheterocyclyl group, an aryl group, a (C₁-C₈)alkylenaryl group, or a (C₂-C₈)-alkenylenaryl group, are another subject of the invention.

Stereoisomers of general formula I according to one of the claims, in which R⁵ means a (C₁-C₅)-alkyl group or an optionally partially or completely fluorinated (C₁-C₅)-alkyl group, an aryl group, a (C₁-C₈)alkylenaryl group, a (C₂-C₈)alkenylenaryl group, a (C₃-C₇)cycloalkyl group, a (C₁-C₈)alkylene(C₃-C₇)cycloalkyl group, or a (C₂-C₅)alkenylene(C₃-C₇)cycloalkyl group are a special subject of the invention.

Stereoisomers of general formula I according to one of the claims, in which R⁵ means a (C₁-C₃)-alkyl group or an optionally partially or completely fluorinated (C₁-C₃)-alkyl group, especially preferably a completely fluorinated (C₁-C₃)alkyl group, or quite especially preferably a CF₃ group, are another subject of the invention.

R⁶ and R⁷, independently of one another, can mean a hydrogen atom, a methyl or ethyl group or, together with the carbon atom of the 5H-benzocycloheptene system, a (C₃-C₆)-cycloalkyl ring. A hydrogen atom, or a methyl or ethyl group is preferred; a methyl or ethyl group is especially preferred; and a methyl group is quite especially preferred.

All combinations of the radicals that are disclosed in the examples are a special subject of the invention.

Definitions

The C₁-C₁₀— or C₁-C₅-alkyl groups can be straight-chain or branched and stand for a methyl, ethyl, n-propyl, iso-propyl-, n-butyl, iso-butyl, tert-butyl- or n-pentyl-, 2,2-dimethylpropyl, 2-methylbutyl or 3-methylbutyl group. A methyl or ethyl group is preferred.

They can optionally be substituted by 1-3 hydroxy groups and/or 1-3 COOR¹⁰ groups. Hydroxy groups are preferred.

For a partially or completely fluorinated C₁-C₅-alkyl group, the following partially or completely fluorinated groups are considered: fluoromethyl, difluoromethyl, trifluoromethyl, fluoroethyl, 1,1-difluoroethyl, 1,2-difluoroethyl, 1,1,1-trifluoroethyl, tetrafluoroethyl, and pentafluoroethyl. Of the latter, the trifluoromethyl group or the pentafluoroethyl group is preferred. A completely fluorinated alkyl group is also called a perfluoroalkyl group, as is known to one skilled in the art.

The C₁-C₁₀— or C₁-C₅-alkoxy groups can be straight-chain or branched and stand for a methoxy, ethoxy, n-propoxy, iso-propoxy, n-butoxy, iso-butoxy, tert-butoxy or n-pentoxy, 2,2-dimethylpropoxy, 2-methylbutoxy or 3-methylbutoxy group. A methoxy or ethoxy group is preferred.

The C₁-C₅-alkylthio groups can be straight-chain or branched and stand for a methylthio, ethylthio, n-propylthio, iso-propylthio, n-butylthio, iso-butylthio, tert-butylthio or n-pentylthio, 2,2-dimethylpropylthio, 2-methylbutylthio or 3-methylbutylthio group. A methylthio or ethylthio group is preferred.

The designation halogen atom or halogen means a fluorine, chlorine, bromine or iodine atom. A fluorine, chlorine, or bromine atom is preferred.

A (C₃-C₇)-cycloalkyl group optionally is defined by one or more groups selected from hydroxy groups, halogen atoms, (C₁-C₅)-alkyl groups, (C₁-C₅)-alkoxy groups, NR⁸R⁹ groups, COOR¹⁰ groups, CHO, cyano, or substituted saturated cyclic groups with 3 to 7 ring-carbon atoms, such as, for example, cyclopropyl, methylcyclopropyl, cyclobutyl, methylcyclobutyl, cyclopentyl, methylcyclopentyl, cyclohexyl, methylcyclohexyl, cycloheptyl, or methylcycloheptyl.

A (C₁-C₈)alkylene(C₃-C₇)cycloalkyl group is, for example, —(CH₂)cycloalkyl, —(C₂H₄)-cycloalkyl, —(C₃H₆)-cycloalkyl, —(C₄H₈)-cycloalkyl, (C₅H₁₀)-cycloalkyl. methylene cyclopropyl, methylene cyclobutyl, methylene cyclopentyl, methylene cyclohexyl, or methylene cycloheptyl.

The (C₂-C₈)alkenylene(C₃-C₇)cycloalkyl group means, for example, —(CH═CH)-cycloalkyl, —[C(CH₃)═CH]-cycloalkyl, —[CH═C(CH₃)]-cycloalkyl, —(CH═CH—CH₂)-cycloalkyl, —(CH₂—CH═CH)-cycloalkyl, —(CH═CH—CH₂—CH₂)-cycloalkyl, —(CH₂—CH═CH—CH₂)-cycloalkyl, —(CH₂—CH₂—CH═CH)-cycloalkyl, —(C(CH₃)═CH—CH₂)-cycloalkyl, or —(CH═C(CH₃)—CH₂)-cycloalkyl.

The heterocyclyl group is a 5- to 7-membered, non-aromatic ring, which contains 1-3 heteroatoms that are selected from nitrogen, oxygen and sulfur as ring members, whereby at the same time, no more than one oxygen or sulfur atom is allowed as a ring member. It can be, for example, pyrrolidine, imidazolidine, pyrazolidine, or piperidine.

Alkylene heterocyclyl groups are defined as heterocyclyl groups that are bonded via a C₁-C₈-alkylene group to the skeleton, whereby the alkylene group can be straight-chain or branched.

If a skeleton is mentioned, the bicyclic bicycloheptene ring system of general formula I is thus meant.

Alkenylene heterocyclyl groups are heterocyclyl groups that are bonded via an unsaturated C₂-C₈-alkylene group to the skeleton, whereby the alkenylene groups can be straight-chain or branched.

In terms of the invention, aryl groups are the aromatic or partially aromatic carbocyclic groups with 6 to 14 carbon atoms that have a ring, such as, e.g., phenyl or phenylene, or several condensed rings, such as, e.g., naphthyl or anthranyl. By way of example, phenyl, naphthyl, tetralinyl, anthranyl, indanyl, and indenyl can be mentioned.

The aryl groups can be substituted at any suitable site that results in a stable compound by one or more radicals from the groups of hydroxy, halogen, C₁-C₅-alkyl, C₁-C₅-alkoxy, cyano, CF₃, or nitro.

As substituents, for example, methoxy, ethoxy, propoxy, iso-propoxy, hydroxy, fluorine, chlorine, bromine, methyl, ethyl, propyl, iso-propyl or trifluoromethyl groups can be mentioned.

The optionally substituted phenyl group and the naphthyl group are preferred.

An alkylenaryl group is an aryl group that is linked via a C₁-C₈-alkylene group to the ring system, whereby the alkenylene group can be straight-chain or branched, and optionally also can carry several double bonds.

An alkenylenaryl group is an aryl group that is bonded via a C₂-C₈-alkenylene group to a skeleton, whereby the alkenylene group can be straight-chain or branched.

The alkinylenaryl group is an aryl group that is bonded via a C₂-C₈-alkinylene group to the skeleton, whereby the alkinylene group can be straight-chain or branched.

A monocyclic or bicyclic heteroaryl group, which can be hydrogenated at one or more sites, is defined as all monocyclic or bicyclic aromatic ring systems that contain at least one heteroatom and at most seven heteroatoms. Ring systems with 1-5 heteroatoms are preferred. As heteroatoms, 1-4 nitrogen atoms, 1-2 oxygen atoms and 1-2 sulfur atoms, which can occur in all sub-combinations in the ring system, are suitable, as long as they do not exceed the number specified for the respective heteroatom and the total maximum number of seven heteroatoms; ring systems that contain 1-3 nitrogen atoms and/or an oxygen atom or a sulfur atom are especially preferred. For example, compounds of formula I, in which R³ or R⁵ means furanyl, thienyl, pyrazolyl, pyrrolyl, oxazolyl, thiazolyl, imidazolyl, isoxazolyl, isothiazolyl, oxadiazolyl, triazolyl, tetrazolyl, thiadiazolyl, pyridyl, pyrimidinyl, pyridazinyl, pyrazinyl, triazinyl, azaindolizinyl, phthalidyl, thiophthalidyl, indolyl, isoindolyl, dihydroindolyl, dihydroisoindolyl, indazolyl, benzothiazolyl, indolonyl, dihydroindolonyl, isoindolonyl, dihydroisoindolonyl, benzofuranyl, benzimidazolyl, indolizinyl, isobenzofuranyl, azaindolyl, azaisoindolyl, furanopyridyl, furanopyrimidinyl, furanopyrazinyl, furanopyridazinyl, dihydrobenzofuranyl, dihydrofuranopyridyl, dihydrofuranopyrimidinyl, dihydrofuranopyrazinyl, dihydrofuranopyridazinyl, dihydrobenzofuranyl, coumarinyl, isocoumarinyl, dihydroisoquinolinyl, dihydroquinolinyl, benzoxazinonyl, phthalazinonyl, quinolinyl, isoquinolinyl, quinolonyl, isoquinolonyl, quinazolinyl, quinoxalinyl, cinnolinyl, phthalazinyl, 1,7- or 1,8-naphthyridinyl, are thus a part of this invention and represent a special embodiment of the invention.

If the heteroaryl groups are partially or completely hydrogenated, compounds of formula I in which R³ or R⁵ means tetrahydropyranyl, 2H-pyranyl, 4H-pyranyl, piperidyl, tetrahydropyridyl, dihydropyridyl, 1H-pyridin-2-onyl, 1H-pyridin-4-onyl, 4-aminopyridyl, 1H-pyridin-4-ylidenaminyl, chromanyl, isochromanyl, chromenyl, isochromenyl, thiochromanyl, decahydroquinolinyl, tetrahydroquinolinyl, dihydroquinolinyl, 5,6,7,8-tetrahydro-1H-quinolin-4-onyl, decahydroisoquinolinyl, tetrahydroisoquinolinyl, dihydroisoquinolinyl, 3,4-dihydro-2H-benz[1,4]oxazinyl, 1,2-dihydro[1,3]benzoxazin-4-onyl, 3,4-dihydrobenz[1,4]oxazin-4-onyl, 3,4-dihydro-2H-benz[1,4]thiazinyl, 4H-benzo[1,4]thiazinyl, 1,2,3,4-tetrahydroquinoxalinyl, 1H-cinnolin-4-onyl, 3H-quinazolin-4-onyl, 1H-quinazolin-4-onyl, 3,4-dihydro-1H-quinoxalin-2-onyl, 2,3-1,2,3,4-tetrahydro [1,5]naphthyridinyl, dihydro-1H-[1,5]naphthyridyl, 1H-[1,5]naphthyrid-4-onyl, 5,6,7,8-tetrahydro-1H-naphthyridin-4-onyl, 1,2-dihydropyrido[3,2-d][1,3]oxazin-4-onyl, octahydro-1H-indolyl, 2,3-dihydro-1H-indolyl, octahydro-2H-isoindolyl, 1,3-dihydro-2H-isoindolyl, 1,2-dihydroindazolyl, 1H-pyrrolo[2,3-b]pyridyl, 2,3-dihydro-1H-pyrrolo[2,3-b]pyridyl, and 2,2-dihydro-1H-pyrrolo[2,3-b]pyridin-3-onyl, are thus part of this invention.

The compounds according to the invention can also be present in the form of salts with physiologically compatible anions, for example in the form of hydrochloride, sulfate, nitrate, phosphate, pivalate, maleate, fumarate, tartrate, benzoate, mesylate, citrate or succinate.

Similar compounds, which metabolize in the organism to form compounds of general formula I according to claim 1, are part of the subject of this invention.

The compounds according to the invention are produced by the open-chain precursors of general formula II being generated according to the methods that are known in the prior art,

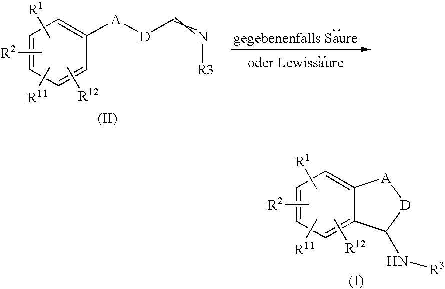

### [Optionally Acid or Lewis Acid]

which then are cyclized by adding inorganic or organic acids or Lewis acids under temperatures in the range of −30° C. to +80° C. to form the compounds of general formula I.

If compounds of general formula I are to be produced, in which R¹ means a (C₁-C₅)alkoxy group, first the halogen compounds must be produced, and then the halogen atom is exchanged for a hydroxy group—according to methods that are known to one skilled in the art by substitution reaction on the aromatic compound—which optionally can be converted into an alkoxy group according to methods that are known to one skilled in the art.

The compounds of general formula II are produced based on the meaning of A according to various methods:

I. Production of the Compounds of General Formula I in Which A Means —CH₂—(CR⁶R⁷): (=Compounds of General Formula Ia):

A compound of general formula III, in which LG means any leaving group that is known to one skilled in the art, such as, e.g., chloride, bromide, iodide, sulfate, sulfonate, e.g., tosylate, mesylate, is reacted under basic conditions, e.g., aqueous NaOH or KOH in organic solvents, such as, e.g., benzene, toluene with an aldehyde of general formula IV to form compounds of general formula V and phase transfer catalysis with commonly used phase transfer catalysts, such as, for example, quaternary ammonium salts, e.g., tetrabutylammonium iodide, benzyltrimethylammonium chloride, to form compounds of general formula V

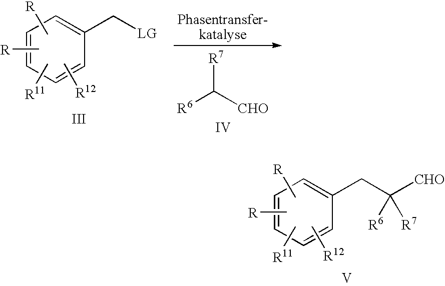

### [Phase Transfer Catalysis]

The aldehyde V is then reacted under Horner-Wittig conditions with the reagent VI, whereby R^{x} means a (C₁-C₃)-alkyl group or a benzyl group, and the compound of general formula VII is obtained. After the ester is saponified according to the method known to one skilled in the art,

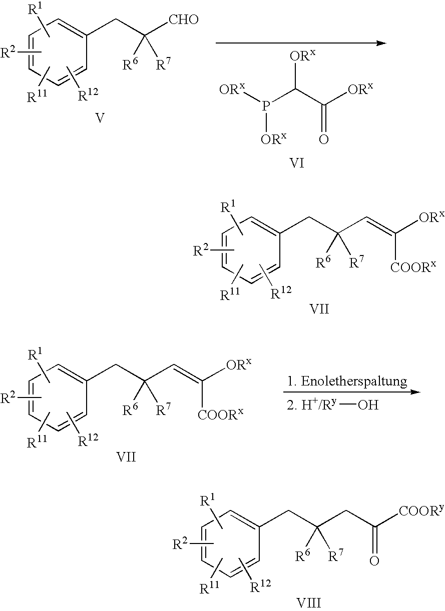

### [Enol Ether Cleavage]

the enol ether is cleaved under acidic conditions, for example glacial acetic acid/HOAc/H₂SO₄, and the x-keto acid that is obtained is esterified under commonly used acidic conditions with an alcohol R^{y}—OH, whereby R^{y} means (C₁-C₅)-alkyl, to form an α-keto ester of general formula VIII. With Ruppert's reagent, CF₃S I(CH₃)₃,

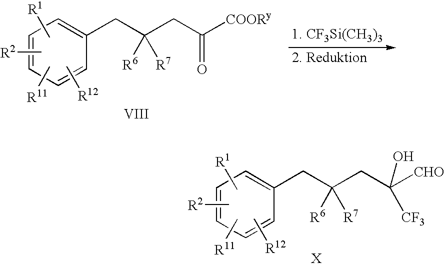

### [Reduction]

the CF₃ group is introduced. With reducing agents, such as, for example, diisobutylaluminum hydride (DIBAH=DIBAL) or lithium aluminum hydride (LAH=LiAlH₄), the ester is reduced to form aldehyde X. The aldehyde is then reacted, as already described in the prior art, with an amine of general formula R³—NH₂, whereby R³ has the meaning that is indicated in the claims.

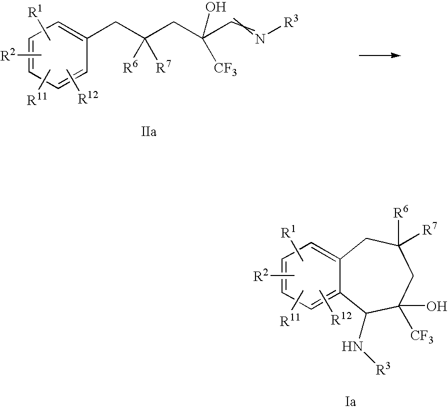

The thus obtained imine Ia is then cyclized to form the compounds of general formula 1a with the addition of Lewis acids at temperatures in the range of −70° C. to +80° C. (preferably in the range of −30° C. to +80° C.) within up to 14 days.

II. Production of the Compounds of General Formula I in Which A Means —(CR⁶R⁷)—CH₂— (=Compounds of General Formula Ib):

A nitrile of general formula XI is optionally reacted in succession with sodium hydride and the corresponding alkyl halides R⁶-Hal and/or R⁷-Hal or Hal-[(C₂-C₅)-alkylene]-Hal to form a compound of general formula XII.

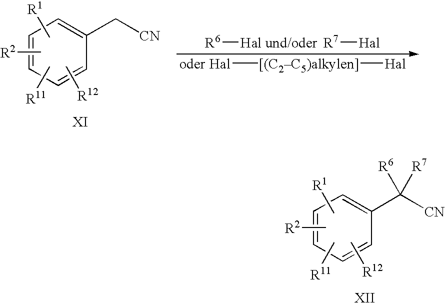

### [R⁶-Hal and/or R⁷-Hal or Hal-[(C₂-C₅)alkylene]-Hal]

The compound of general formula XII that is obtained is reduced under common conditions with inhibited hydrides, such as, for example, diisobutylaluminum hydride (DIBAH, DIBAL) to form aldehyde

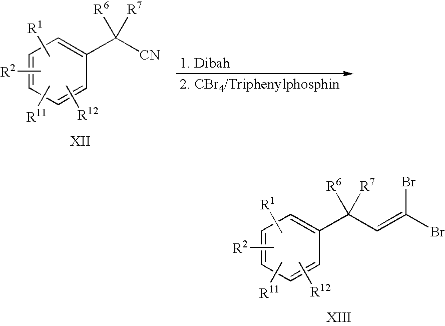

### [ . . . Triphenylphosphine]

and then is reacted with CBr₄/triphenylphosphine optionally with the addition of zinc to form a compound of general formula XIII.

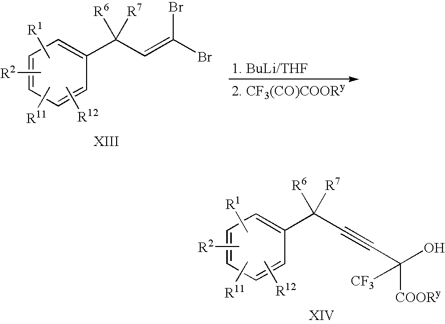

With butyllithium, the compound of general formula XIII is converted into the acetylene and with CF₃—(CO)COOR^{y} into the ester XIV. The ester is hydrogenated according to methods known to one skilled in the art to form esters of general formula

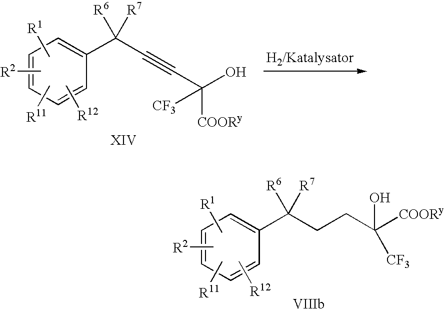

### [H₂/Catalyst]

VIIIb, which then is reduced analogously to process I to form aldehyde, is reacted with the amine R³—NH₂ to form imine, and then is cyclized to

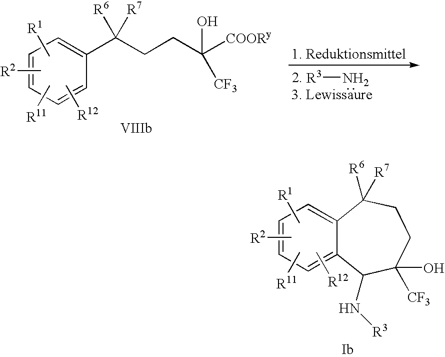

### [Reducing Agent]

### [Lewis Acid]

the compound of general formula Ib.

As Lewis acids, for example, BBr₃, TiCl₄, Ti(OR³)₄, TiCl₂(OR³)₂, TiBr₂(OR³)₂, PdCl₄, Pd(OR³)₄, PdCl₂(OR³)₂, PdBr₂(OR³)₂, ZnCl₂, ZnBr₂, AlCl₃, AlBr₃, AlEtCl₂, Al Me₂Cl, Cu salts, e.g., Cu(OTf)₂, CuCl₂, CuBr₂, and Yb(OTf)₃ are suitable.

When using chiral Lewis acids, for example, the following Lewis acids are suitable: (R)- or (S)-SEGPHOS-PdCl₂ (Mikami et al. Tetrah. Asymm. 2004, 15, 3885-89), (R)- or (S)-BINOL-Ti(OiPr)₂ (Ding et al. Tetrah. Lett. 2004, 45, 2009-12), (R)- or (S)-Cu ^{t}BuBOX, (R)- or (S)-Cu ^{t}PrBOX, (R)- or (S)-Cu PhBOX, (R)- or (S)-Cu AdaBOX (Evans et al., J. Am. Chem. Soc. 2000, 122, 7936-43), (R)- or (S)-Ph-pybox Sc (OTf)₃ (Evans et al. J. Am. Chem. Soc. 2005, 127, 8006-7), (R)- or (S)-'Pr-pybox Yb(OTf)₃, (R)- or-(S)-^{t}Bu-pybox Yb (OTf)₃, (R)- or (S)-Ph-pybox Yb (OTf)₃ (Qian et al. Tetrah. Asymm. 2000, 11, 2347-57).

The new intermediate products of the cited syntheses are also subjects of this invention.

The binding of substances to the glucocorticoid receptor (GR) and other steroid hormone receptors (mineral corticoid receptors (MR), progesterone receptors (PR) and androgen receptors (AR)) is examined with the aid of recombinantly produced receptors. Cytosol preparations of Sf9 cells that had been infected with recombinant baculoviruses, which code for the GR, are used for the binding studies. In comparison to the reference substance [³H]-dexamethasone, the substances show a high affinity to the GR.

The GR-mediated inhibition of the transcription of cytokines, adhesion molecules, enzymes and other pro-inflammatory factors is considered to be an essential, molecular mechanism for the anti-inflammatory action of glucocorticoids. This inhibition is produced by interaction of GR with other transcription factors, e.g., AP-1 and NF-kappa-B (for an overview, see Cato, A. C. B. and Wade, E., BioEssays 18, 371-378 1996).

The compounds of general formula I according to the invention inhibit the secretion of cytokine IL-8, triggered by lipopolysaccharide (LPS), in the human monocyte cell line THP-1. The concentration of the cytokines was determined in the supernatant by means of commercially available ELISA kits.

The anti-inflammatory action of the compounds of general formula I was tested in the animal experiment by tests in the croton oil-induced inflammation in rats and mice (J. Exp. Med. (1995),182, 99-108). To this end, croton oil in ethanolic solution was applied topically to the animals' ears. The test substances were also applied topically or systemically at the same time or two hours before the croton oil. After 16-24 hours, the ear weight was measured as a measurement of inflammatory edema, the peroxidase activity as a measurement of the invasions of granulocytes, and the elastase activity as a measurement of the invasion of neutrophilic granulocytes. In this test, the compounds of general formula I inhibit the three above-mentioned inflammation parameters both after topical and after systemic administration.

One of the most frequent undesirable actions of a glucocorticoid therapy is the so-called “steroid diabetes” [cf. Hatz, H. J., Glucocorticoide: Immunologische Grundlagen, Pharmakologie and Therapierichtlinien [Glucocorticoids: Immunological Principles, Pharmacology and Therapy Guidelines], Wissenschaftliche Verlagsgesellschaft mbH, Stuttgart, 1998]. The reason for this is the stimulation of gluconeogenesis in the liver by induction of the enzymes responsible in this respect and by free amino acids that are produced from the degradation of proteins (catabolic action of glucocorticoids). One key enzyme of the catabolic metabolism in the liver is the tyrosinaminotransferase (TAT). The activity of this enzyme can be determined by photometry from liver homogenates and represents a good measurement of the undesirable metabolic actions of glucocorticoids. To measure the TAT induction, the animals are sacrificed 8 hours after the test substances are administered, the livers are removed, and the TAT activity in the homogenate is measured. In this test, at doses in which they have an anti-inflammatory action, the compounds of general formula I do not induce the tyrosinaminotransferase, or induce it to only a small extent.

Based on their anti-inflammatory and additional anti-allergic, immunosuppressive and anti-proliferative action, the compounds of general formula I according to the invention can be used as medications for treatment or prophylaxis of the following pathologic conditions in mammals and humans: In this case, the term “DISEASE” stands for the following indications:

(i) Lung diseases that are accompanied by inflammatory, allergic and/or proliferative processes:

Chronic, obstructive lung diseases of any origin, primarily bronchial asthma
Bronchitis of different origins
Adult respiratory distress syndrome (ARDS)
Bronchiectases
All forms of restrictive lung diseases, primarily allergic alveolitis,
All forms of pulmonary edema, primarily toxic pulmonary edema; e.g., radiogenic pneumonitis
Sarcoidoses and granulomatoses, especially Boeck's disease
   (ii) Rheumatic diseases/autoimmune diseases/joint diseases that are accompanied by inflammatory, allergic and/or proliferative processes:
All forms of rheumatic diseases, especially rheumatoid arthritis, acute rheumatic fever, polymyalgia rheumatica, Behçet's disease
Reactive arthritis
Inflammatory soft-tissue diseases of other origins
Arthritic symptoms in the case of degenerative joint diseases (arthroses)
Traumatic arthritides
Vitiligo
Collagenoses of any origin, e.g., systemic lupus erythematodes, sclerodermia, polymyositis, dermatomyositis, Sjögren's syndrome, Still's syndrome, Felty's syndrome
Sarcoidoses and granulomatoses
Soft-tissue rheumatism
   (iii) Allergies or pseudoallergic diseases that are accompanied by inflammatory and/or proliferative processes:
All forms of allergic reactions, e.g., Quincke's edema, hay fever, insect bites, allergic reactions to pharmaceutical agents, blood derivatives, contrast media, etc., anaphylactic shock, urticaria, allergic and irritative contact dermatitis, allergic vascular diseases
Allergic vasculitis
   (iv) Vascular inflammations (vasculitides)
Panarteritis nodosa, temporal arteritis, erythema nodosum
Polyarteris nodosa
Wegner's granulomatosis
Giant-cell arteritis
   (v) Dermatological diseases that are accompanied by inflammatory, allergic and/or proliferative processes:
Atopic dermatitis (primarily in children)
All forms of eczema, such as, e.g., atopic eczema (primarily in children)
Rashes of any origin or dermatoses
Psoriasis and parapsoriasis groups
Pityriasis rubra pilaris
Erythematous diseases, triggered by different noxae, e.g., radiation, chemicals, burns, etc.
Bullous dermatoses, such as, e.g., autoimmune pemphigus vulgaris, bullous pemphigoid
Diseases of the lichenoid group,
Pruritis (e.g., of allergic origin)
Seborrheal eczema
Rosacea group
Pemphigus vulgaris
Erythema exudativum multiforme
Balanitis
Vulvitis
Manifestation of vascular diseases
Hair loss such as alopecia areata
Cutaneous lymphoma
Parapsoriasis
   (vi) Kidney diseases that are accompanied by inflammatory, allergic and/or proliferative processes:
Nephrotic syndrome
All nephritides, e.g., glomerulonephritis
   (vii) Liver diseases that are accompanied by inflammatory, allergic and/or proliferative processes:
Acute liver cell decomposition
Acute hepatitis of different origins, e.g., viral, toxic, pharmaceutical agent-induced
Chronic aggressive hepatitis and/or chronic intermittent hepatitis
   (viii) Gastrointestinal diseases that are accompanied by inflammatory, allergic and/or proliferative processes:
Regional enteritis (Crohn's disease)
Colitis ulcerosa
Gastritis
Reflux esophagitis
Ulcerative colitis of other origins, e.g., native sprue
   (ix) Proctologic diseases that are accompanied by inflammatory, allergic and/or proliferative processes:
Anal eczema
Fissures
Hemorrhoids
Idiopathic proctitis
   (x) Eye diseases that are accompanied by inflammatory, allergic and/or proliferative processes:
Allergic keratitis, uveitis, iritis
Conjunctivitis
Blepharitis
Optic neuritis
Chorioiditis
Sympathetic ophthalmia
   (xi) Diseases of the ear-nose-throat area that are accompanied by inflammatory, allergic and/or proliferative processes:
Allergic rhinitis, hay fever
Otitis externa, e.g., caused by contact dermatitis, infection, etc.
Otitis media
   (xii) Neurological diseases that are accompanied by inflammatory, allergic and/or proliferative processes:
Cerebral edema, primarily tumor-induced cerebral edema
Multiple sclerosis
Acute encephalomyelitis
Meningitis
Various forms of convulsions, e.g., infantile nodding spasms
Acute spinal cord injury
Stroke
   (xiii) Blood diseases that are accompanied by inflammatory, allergic and/or proliferative processes, such as, e.g.:
Acquired hemolytic anemia
Idiopathic thrombocytopenia
   (xiv) Tumor diseases that are accompanied by inflammatory, allergic and/or proliferative processes, such as, e.g.:
Acute lymphatic leukemia
Malignant lymphoma
Lymphogranulomatoses
Lymphosarcoma
Extensive metastases, mainly in breast, bronchial and prostate cancers
   (xv) Endocrine diseases that are accompanied by inflammatory, allergic and/or proliferative processes, such as, e.g.:
Endocrine orbitopathy
Thyreotoxic crisis
De Quervain's thyroiditis
Hashimoto's thyroiditis
Basedow's disease
Endocrine ophthalmopathy
Granulomatous thyroiditis
Lymphadenoid goitre
Graves' disease
   (xvi) Organ and tissue transplants, graft-versus-host disease
   (xvii) Severe shock conditions, e.g., anaphylactic shock, systemic-inflammatory response syndrome (SIRS)
   (xviii) Substitution therapy in:
Innate primary suprarenal insufficiency, e.g., congenital adrenogenital syndrome
Acquired primary suprarenal insufficiency, e.g., Addison's disease, autoimmune adrenalitis, meta-infective tumors, metastases, etc.
Innate secondary suprarenal insufficiency, e.g., congenital hypopituitarism
Acquired secondary suprarenal insufficiency, e.g., meta-infective tumors, etc.
   (xix) Vomiting that is accompanied by inflammatory, allergic and/or proliferative processes:
e.g., in combination with a 5-HT3 antagonist in cytostatic-agent-induced vomiting
   (xx) Pains of inflammatory origins, e.g., lumbago
   (xxi) Various diseases.

The local administration of the compounds according to the invention for treatment of diseases that are cited under the items (i), (ii), (iii), (v), (viii), (ix), (x), (xi), (xv), (xx) and (xxi), in particular (i), (ii), (iii), (v), (x), (xi) and (xv), is preferred.

Moreover, the compounds of general formula I according to the invention can be used for treatment and prophylaxis of additional pathologic conditions that are not mentioned above, for which synthetic glucocorticoids are now used (see in this respect Hatz, H. J., Glucocorticoide: Immunologische Grundlagen, Pharmakologie und Therapierichtlinien, Wissenschaftliche Verlagsgesellschaft mbH, Stuttgart, 1998).

All previously mentioned indications (i) to (xx) are described in more detail in Hatz, H. J., Glucocorticoide: Immunologische Grundlagen, Pharmakologie und Therapierichtlinien, Wissenschaftliche Verlagsgesellschaft mbH, Stuttgart, 1998.

The invention also relates to combination therapies or combined compositions, in which a glucocorticoid receptor (GR) agonist of formula (I) or a pharmaceutically acceptable salt thereof, or a pharmaceutical composition that contains a GR agonist of formula (I) or a pharmaceutically acceptable salt thereof, is administered either simultaneously (optionally in the same composition) or in succession together with one or more pharmaceutical agents for treating one of the above-mentioned pathologic conditions. For example, for treatment of rheumatoid arthritis, osteoarthritis, COPD (chronic obstructive lung disease), asthma or allergic rhinitis, a GR agonist of this invention can be combined with one or more pharmaceutical agents for treating such a condition. When such a combination is administered by inhalation, the pharmaceutical agent that is to be combined can be selected from the following list:
A PDE4 inhibitor including an inhibitor of the PDE4D isoform,
A selective β.sub2.adrenoceptor agonist, such as, for example, metaproterenol, isoproterenol, isoprenaline, albuterol, salbutamol, formoterol, salmeterol, terbutaline, orcipresnaline, bitolterol mesylate, pirbuterol or indacaterol;
A muscarine receptor antagonist (for example, an M1, M2 or M3 antagonist, such as, for example, a more selective M3 antagonist), such as, for example, ipratropium bromide, tiotropium bromide, oxitropium bromide, pirenzepine or telenzepine;
A modulator of the chemokine receptor function (such as, for example, a CCR1 receptor antagonist); or
An inhibitor of the p38 kinase function.

For another subject of this invention, such a combination with a GR agonist of formula (I) or a pharmaceutically acceptable salt thereof is used for treating COPD, asthma or allergic rhinitis and can be administered by inhalation or orally in combination with xanthine (such as, for example, aminophylline or theophylline), which also can be administered by inhalation or orally.

For the therapeutic actions in the above-mentioned pathologic conditions, the suitable dose varies and depends on, for example., the active strength of the compound of general formula I, the host, the type of administration, and the type and severity of the conditions that are to be treated, as well as the use as a prophylactic agent or therapeutic agent.

In addition, the invention provides:
(i) The use of one of the compounds of formula I according to the invention or mixture thereof for the production of a medication for treating a DISEASE;
(ii) A process and a method for treating a DISEASE, said process comprises an administration of an amount of the compound according to the invention whereby the amount suppresses the disease and whereby the amount of the compound is given to a patient who requires such a medication;
(iii) A pharmaceutical composition for treating a DISEASE, said treatment comprises one of the compounds according to the invention or mixture thereof and at least one pharmaceutical adjuvant and/or vehicle.

In general, satisfactory results can be expected in animals when the daily doses comprise a range of 1 μg to 100,000 μg of the compound according to the invention per kg of body weight. In the case of larger mammals, for example the human, a recommended daily dose lies in the range of 1 μg to 100,000 μg per kg of body weight. Preferred is a dose of 10 to 30,000 μg per kg of body weight, and more preferred is a dose of 10 to 10,000 μg per kg of body weight. For example, this dose is suitably administered several times daily. To treat acute shock (e.g., anaphylactic shock), single doses that clearly exceed the above-mentioned doses can be given.

The formulation of the pharmaceutical preparations based on the new compounds is carried out in a way that is known in the art by the active ingredient being processed with the vehicles, fillers, substances that influence decomposition, binding agents, moisturizers, lubricants, absorbents, diluents, flavoring correctives, coloring agents, etc., that are commonly used in galenicals and converted into the desired form of administration. In this case, reference is made to Remington's Pharmaceutical Science, 15^{th} Edition, Mack Publishing Company, East Pennsylvania (1980).

For oral administration, especially tablets, coated tablets, capsules, pills, powders, granulates, lozenges, suspensions, emulsions or solutions are suitable.

For parenteral administration, injection and infusion preparations are possible.

For intra-articular injection, correspondingly prepared crystal suspensions can be used.

For intramuscular injection, aqueous and oily injection solutions or suspensions and corresponding depot preparations can be used.

For rectal administration, the new compounds can be used in the form of suppositories, capsules, solutions (e.g., in the form of enemas) and ointments both for systemic and for local treatment.

For pulmonary administration of the new compounds, the latter can be used in the form of aerosols and inhalants.

For local application to eyes, outer ear channels, middle ears, nasal cavities, and paranasal sinuses, the new compounds can be used as drops, ointments and tinctures in corresponding pharmaceutical preparations.

For topical application, formulations in gels, ointments, fatty ointments, creams, pastes, powders, milk and tinctures are possible. The dosage of the compounds of general formula I should be 0.01%-20% in these preparations to achieve a sufficient pharmacological action.

The invention also comprises the compounds of general formula I according to the invention as therapeutic active ingredients. In addition, the compounds of general formula I according to the invention are part of the invention as therapeutic active ingredients together with pharmaceutically compatible and acceptable adjuvants and vehicles.

The invention also comprises a pharmaceutical composition that contains one of the pharmaceutically active compounds according to the invention or mixture thereof or pharmaceutically compatible salt thereof and a pharmaceutically compatible salt or pharmaceutically compatible adjuvants and vehicles.

Experimental Part

### EXAMPLE 1

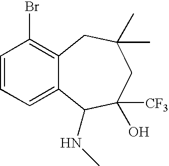

### 1-Bromo-6,7,8,9-tetrahydro-5-[(1H-indazol-4-yl)amino]-8,8-dimethyl-6-(trifluoromethyl)-5H-benzocyclohepten-6-ol

### 3-(2-Bromophenyl)-2,2-dimethylpropanal

1.63 g (40.81 mmol) of triturated sodium hydroxide and 150.7 mg (0.41 mmol) of tetrabutylammonium iodide are introduced into six milliliters of benzene. A mixture that consists of 12.7 g (51 mmol) of 2-bromobenzyl bromide and 2.94 g (40.81 mmol) of isobutyraldehyde are added in drops to this mixture at 60° C., and the batch is then stirred for eight hours at this temperature. After stirring overnight at room temperature, the reaction mixture is mixed with 40 ml of water and shaken with 400 ml of diethyl ether. The organic phase is washed with water and saturated sodium chloride solution. After drying on sodium sulfate, the solvent is spun off, and the remaining residue is chromatographed on silica gel (mobile solvent ethyl acetate/hexane). 6.6 g (67.1%) of the desired compound is isolated.

MS (EI) m/e (relative intensity): 242 (M⁺, 3), 169/71 (100)

### (E/Z)-Ethyl-5-(2-bromophenyl)-4,4-dimethyl-2-ethoxypent-2-enoate

14.75 ml of a 2 M solution of lithium diisopropylamide in tetrahydrofuran/heptane/toluene is added in drops to a solution of 7.34 g (23.371 mmol) of 2-diethylphosphono-2-ethoxyacetic acid ethyl ester in 21 ml of tetrahydrofuran while being slowly cooled with ice, and it is stirred for 30 minutes at 0° C. 6.6 g (27.37 mmol) of 3-(2-bromophenyl)-2,2-dimethyl-propanal, dissolved in 31.5 ml of anhydrous tetrahydrofuran, is added in drops at 0° C. to this yellow solution. The cold bath is removed, and the batch is stirred for 22 hours at room temperature. The reaction mixture is mixed with 60 ml of water and shaken twice with 300 ml each of diethyl ether. The combined organic extracts are washed with water and saturated sodium chloride solution. After drying on sodium sulfate and after the solvent has spun off, the remaining residue is chromatographed on silica gel (mobile solvent ethyl acetate/hexane). 6.63 g (68.2%) of the desired compound, specifically as an E/Z mixture, is isolated.

¹H-NMR (300 MHz, CDCl₃): δ=1.10-1.40 (12H), 2.95-3.08 (2H), 3.68-3.80 (2H), 4.13-4.32 (2H), 4.89 and 6.17 (together 1H), 7.00-7.10 (1H), 7.14-7.34 (2H), 7.55 (1H).

### 5-(2-Bromophenyl)-4,4-dimethyl-2-oxopentanoic Acid

6.63 g (18.66 mmol) of (E/Z)-ethyl-5-(2-bromophenyl)-4,4-dimethyl-2-ethoxypent-2-enoate is mixed with 186 ml of 1 M sodium hydroxide solution in a 2:1 mixture that consists of ethanol/water, and it is stirred for 18 hours at room temperature. After the ethanol is drawn off, the residue is extracted with diethyl ether. The organic phase is discarded. The aqueous phase is acidified with concentrated HCl until a pH of 3 is reached. After extraction with diethyl ether, the combined organic extracts are washed neutral, dried, and the solvent is spun off. The product that is obtained (5.83 g=95.6%) is used directly in the enol ether cleavage. To this end, the crude product is mixed with 108 ml of a 1 M sulfuric acid and stirred for 17 hours at 90° C. The reaction mixture is mixed with solid potassium carbonate while being cooled in an ice bath until a pH of 9 is reached (strong foaming), and it is extracted with diethyl ether. The ether phase is discarded after being monitored by TLC. The aqueous phase is acidified with concentrated HCl until a pH of 4 is reached. After extraction with diethyl ether, and after the organic phase is washed and dried, the solvent is spun off, and the residue that is obtained (4.23 g=79.8%) is incorporated in crude form into the next stage.

¹H-NMR (300 MHz, CDCl₃): δ=1.10 (6H), 2.94 (2H), 3.00 (2H), 7.05-7.14 (1H), 7.18-7.29 (2H), 7.58 (1H).

### Methyl-5-(2-bromophenyl)-4,4-dimethyl-2-oxopentanoate

2 g (6.685 mmol) of 5-(2-bromophenyl)-4,4-dimethyl-2-oxopentanoic acid is dissolved in 11.6 ml of acetone and mixed at room temperature with 1.108 g (8.022 mmol) of potassium carbonate. After 1.23 g (8.691 mmol) of iodomethane is added, the batch is refluxed for two hours. The potassium carbonate is suctioned off on a G4 frit, washed with acetone, and the solvent is spun off. After the residue is chromatographed on silica gel (mobile solvent ethyl acetate/hexane), 1.1 g (52.6%) of the desired compound is obtained.

¹H-NMR (300 MHz, CDCl₃): δ=1.10 (6H), 2.88 (2H), 2.93 (2H), 3.88 (3H), 7.03-7.11 (1H), 7.18-7.25 (2H), 7.58 (1H).

### 5-(2-Bromophenyl)-4,4-dimethyl-2-(trifluoromethyl)-pentane-1,2-diol

1.1 g (3.512 mmol) of methyl-5-(2-bromophenyl)-4,4-dimethyl-2-oxopentanoate is dissolved in 5.7 ml of THF and mixed at 0° C. with 599.2 mg (4.214 mmol) of (trifluoromethyl)-trimethylsilane. After 8.6 mg of tetrabutylammonium fluoride is added, the batch is stirred for one and one-half hours at 0 to 5° C. The reaction mixture is poured into ice water and extracted twice with diethyl ether. The combined organic extracts are washed with water and brine and dried. After the solvent is spun off, the residue is chromatographed on silica gel (mobile solvent: ethyl acetate/hexane). 1.17 g (73.1%) of methyl-5-(2-bromophenyl)-4,4-dimethyl-2-(trimethylsilanyloxy)-2-(trifluoromethyl)-pentanoate is isolated. The ester is dissolved in 10.7 ml of toluene and mixed at −10° C. with 5.35 ml of a 1.2 M DIBAH solution. After one more hour of stirring at −10 to 0° C., first 1.77 ml of isopropanol and then 2.67 ml of water are carefully added in drops at −10° C. After two hours of vigorous stirring, the precipitate is suctioned off on a frit, washed with ethyl acetate, and the solvent is spun off in a rotary evaporator. 1.07 g of the pentanediol is obtained as a silyl ether, which is incorporated without further purification into the next stage. To this end, the crude product (1.07 g=2.503 mmol) is dissolved in 14 ml of THF and mixed with 789.7 mg (2.503 mmol) of tetrabutylammonium fluoride. After 20 minutes of stirring at room temperature, the batch is added to ice water and extracted twice with diethyl ether. The combined organic extracts are washed with water and with brine. After drying with sodium sulfate, the solvent is spun off, and the residue is chromatographed on silica gel (mobile solvent ethyl acetate/hexane). 582.5 mg (65.5%) of the desired diol is isolated.

¹H-NMR (300 MHz, DMSO-d₆): δ=0.93-1.10 (6H), 1.63 (1H), 1.79 (1H), 2.82-2.98 (2H), 3.52-3.72 (2H), 5.13 (1H), 5.59 (1H), 7.10-7.19 (1H), 7.25-7.40 (2H), 7.59 (1H).

### 5-(2-Bromophenyl)-2-hydroxy-4,4-dimethyl-2-(trifluoromethyl)-pentanal

5.6 ml of dimethyl sulfoxide and 826.2 mg (8.165 mmol) of triethylamine are added to 580 mg (1.633 mmol) of 5-(2-bromophenyl)-4,4-dimethyl-2-(trifluoromethyl)-pentane-1,2-diol, dissolved in 17 ml of dichloromethane. After the addition, in portions, of 779.7 mg (4.899 mmol) of SO₃/pyridine complex, the batch is stirred for 17 hours at room temperature. After 6.5 ml of saturated ammonium chloride solution is added in drops, the batch is stirred for 15 minutes at room temperature and then extracted twice with diethyl ether. The combined organic extracts are washed with water and with brine. After the solvent is dried and spun off, the residue is chromatographed on silica gel (mobile solvent ethyl acetate/hexane). 480.5 mg (83.3%) of the desired compound is isolated.

¹H-NMR (300 MHz, CDCl₃): δ=0.95 (3H), 1.02 (3H), 2.13-2.30 (2H), 2.80-2.93 (2H), 3.93 (1H), 7.02-7.13 (1H), 7.20-7.29 (2H), 7.58 (1H), 9.80 (1H).

### 5-(2-Bromophenyl)-1,1,1-trifluoro-2-[(1H-indazol-4-ylimino)-methyl]-4,4-dimethylpentan-2-ol

100 mg (0.283 mmol) of the above-described aldehyde and 37.7 mg (0.283 mmol) of 4-aminoindazole are mixed with 0.4 ml of glacial acetic acid and stirred for four days at room temperature. The reaction mixture is drawn off three times with toluene in a rotary evaporator, and the residue is then chromatographed (silica gel; mobile solvent ethyl acetate/hexane). 101.8 mg (76.8%) of the desired compound is isolated.

¹H-NMR (300 MHz, CDCl₃): δ=1.02 (3H), 1.18 (3H), 2.09 (1H), 2.39 (1H), 2.98 (2H), 5.10 (1H), 6.82 (1H), 7.00-7.10 (1H), 7.15-7.32 (2H), 7.35-7.49 (2H), 7.56 (1H), 8.15 (1H), 8.23 (1H).

### 1-Bromo-6,7,8,9-tetrahydro-5-[(1H-indazol-4-yl)amino]-8,8-dimethyl-6-(trifluoromethyl)-5H-benzocyclohepten-6-ol

60 mg (0.128 mmol) of 5-(2-bromophenyl)-1,1,1-trifluoro-2-[(1H-indazol-4-ylimino)-methyl]-4,4-dimethylpentan-2-ol is mixed with 2.5 ml of boron tribromide (1M in dichloromethane) and then stirred for seven days at room temperature. The reaction mixture is mixed drop by drop at −30° C. with saturated sodium bicarbonate solution and stirred vigorously for 10 minutes at room temperature after ethyl acetate is added. After two extraction cycles with ethyl acetate, the combined organic extracts are washed with water and brine and dried. After the solvent is spun off, the residue is chromatographed in a Flashmaster. 43.3 mg (70.5%) of the desired compound is isolated.

¹H-NMR (300 MHz, CD₃OD): δ=1.00 (3H), 1.30 (3H), 1.40 (1H), 1.96 (1H), 3.09 (1H), 3.40 (1H), 5.30 (1H), 5.76 (1H), 6.79 (1H), 7.00-7.10 (2H), 7.41 (1H), 7.56 (1H), 8.13 (1H).

### EXAMPLE 2

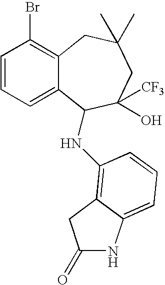

### 4-{[1-Bromo-6,7,8,9-tetrahydro-6-hydroxy-8,8-dimethyl-6-(trifluoromethyl)-5H-benzocyclohepten-5-yl]amino}-1,3-dihydro-2H-indol-2-one and 4-{[1-bromo-6,7,8,9-tetrahydro-6-hydroxy-8,8-dimethyl-6-(trifluoromethyl)-5H-benzocyclohepten-5-yl]amino}-7-bromo-1,3-dihydro-2H-indol-2-one

### 7-{[5-(2-Bromophenyl)-2-hydroxy-4,4-dimethyl-2-(trifluoromethyl)pentylidene]amino}-1,3-dihydro-2H-indol-2-one

73 mg (0.21 mmol) of the 5-(2-bromophenyl)-2-hydroxy-4,4-dimethyl-2-(trifluoromethyl)-pentanal that is described in Example 1 is stirred with 30.62 mg (0.29 mmol) of 4-amino-1,3-dihydro-2H-indol-2-one and 0.29 ml of glacial acetic acid for five days at room temperature. The reaction mixture is drawn off three times with toluene in a rotary evaporator, and the remaining residue is chromatographed on silica gel (mobile solvent hexane/ethyl acetate). 82.9 mg of the desired imine is isolated.

¹H-NMR (300 MHz, CDCl₃): δ=1.00 (3H), 1.10 (3H), 2.02 (1H), 2.33 (1H), 2.95 (2H), 3.55 (2H), 4.90 (1H), 6.69 (1H), 6.83 (1H), 7.08 (1H), 7.18-7.32 (3H), 7.55 (1H), 8.09 (1H), 8.32 (1H).

### 4-{[1-Bromo-6,7,8,9-tetrahydro-6-hydroxy-8,8-dimethyl-6-(trifluoromethyl)-5H-benzocyclohepten-5-yl]amino}-1,3-dihydro-2H-indol-2-one (I) and 4-{(1-bromo-6,7,8,9-tetrahydro-6-hydroxy-8,8-dimethyl-6-(trifluoromethyl)-5H-benzocyclohepten-5-yl]amino}-7-bromo-1,3-dihydro-2H-indol-2-one (II)

81 mg (0.17 mmol) of the above-described imine is mixed at room temperature with 3.4 ml of a 1 M boron tribromide solution in dichloromethane and stirred for 13 days at this temperature. For working-up, the reaction mixture is added to a mixture that consists of saturated sodium bicarbonate solution and ice. After dilution with 50 ml of ethyl acetate, the mixture is stirred vigorously for 20 minutes. The organic phase is separated, and the aqueous phase is shaken another time with 50 ml of ethyl acetate. The combined organic extracts are washed with water and brine and dried on sodium sulfate. After the desiccant is filtered off and after the solvent is spun off, the remaining residue is chromatographed several times on silica gel (amine phase). 21.5 mg (26.5%) of 4-{[1-bromo-6,7,8,9-tetrahydro-6-hydroxy-8,8-dimethyl-6-(trifluoromethyl)-5H-benzocyclohepten-5-yl]amino}-1,3-dihydro-2H-indol-2-one (I) and 7.9 mg (8.4%) of 4-{[1-bromo-6,7,8,9-tetrahydro-6-hydroxy-8,8-dimethyl-6-(trifluoromethyl)-5H-benzocyclohepten-5-yl]amino}-7-bromo-1,3-dihydro-2H-indol-2-one (II) are isolated.

(I): ¹H-NMR (400 MHz, CD₃OD): δ=0.92 (3H), 1.20 (3H), 1.30 (1H), 1.88 (1H), 2.98 (1H), 3.30 (2H, signal is less than the methanol signal), 3.48 (1H), 5.15 (1H), 5.87 (1H), 6.23 (1H), 6.89 (1H), 7.05 (1H), 7.31 (1H), 7.50 (1H).

(II): ¹H-NMR (300 MHz, CD₃OD): δ=0.90 (3H), 1.20 (3H), 1.30 (1H), 1.87 (1H), 2.98 (1H), 3.30 (2H), signal is less than the methanol signal), 3.59 (1H), 5.10 (1H), 5.82 (1H), 6.99 (1H), 7.09 (1H), 7.30 (1H), 7.50 (1H).

### EXAMPLE 3

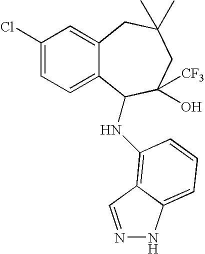

### 2-Chloro-6,7,8,9-tetrahydro-5-(1H-indazol-4-ylamino)-8,8-dimethyl-6-(trifluoromethyl)-5H-benzocyclohepten-6-ol

### 3-(3-Chlorophenyl)-2,2-dimethylpropanol

7.95 g (.198.7 mmol) of triturated sodium hydroxide and 734 mg (1.98 mmol) of tetrabutylammonium iodide are introduced into 38.2 ml of benzene. A mixture that consists of 40 g (248.4 mmol) of 3-chlorobenzyl bromide and 14.33 g (198.7 mmol) of isobutyraldehyde is added in drops to this mixture at 60° C. (heating of the reaction mixture to 72° C.). Then, the batch is stirred for 60 hours at 60° C. After 24 hours of stirring at room temperature, the reaction mixture is mixed with 200 ml of ice water and shaken three times with 200 ml each of methyl-tert-butyl ether. The combined organic phases are washed with water and saturated sodium chloride solution. After drying on sodium sulfate, the solvent is spun off, and the remaining residue is chromatographed on silica gel (mobile solvent ethyl acetate/hexane). 31.74 g (81.2%) of the desired compound (purity about 50%) is isolated.

MS (Cl) m/e (relative intensity): 214 (100%)

### (E/Z)-Ethyl-5-(3-chlorophenyl)-4,4-dimethyl-2-ethoxypent-2-enoate

85.2 ml of a 2 M solution of lithium diisopropylamide in tetrahydrofuran/heptane/toluene is slowly added in drops to a solution of 21.36 g (79.62 mmol) of 2-diethylphosphono-2-ethoxyacetic acid ethyl ester in 100 ml of tetrahydrofuran while being cooled with ice, and it is stirred for 30 minutes at 0° C. 28.46 g (relative to the purity 15.66 g 79.62 mmol) of 3-(3-chlorophenyl-2,2-dimethyl-propanal, dissolved in 100 ml of anhydrous tetrahydrofuran, is added in drops at 0° C. to this yellow solution. The cold bath is removed, and the batch is stirred overnight at room temperature. The reaction mixture is poured into 400 ml of water and shaken three times with 200 ml each of methyl-tert-butyl ether. The combined organic extracts are washed with water and saturated sodium chloride solution. After the sodium sulfate is dried and after the solvent is spun off, the remaining residue is chromatographed on silica gel (mobile solvent ethyl acetate/hexane). 25.76 g (>100%) of the desired compound is isolated as an E/Z mixture.

MS (Cl) m/e (relative intensity): 330 (35%), 328 (100%), 185 (53%).

### 5-(3-CChlorophenyl)-4,4-dimethyl-2-oxopentanoic Acid

28.47 g (91.58 mmol) of (E/Z)-ethyl-5-(3-chlorophenyl)-4,4-dimethyl-2-ethoxypent-2-enoate is mixed with 751 ml of 1 M sodium hydroxide solution in a 2:1 mixture that consists of ethanol/water and stirred overnight at room temperature. After the ethanol is drawn off in a rotary evaporator, the residue is diluted with 200 ml of water and extracted three times with 200 ml each of methyl-tert-butyl ether. The organic phase is discarded. The aqueous phase is acidified with concentrated HCl up to a pH of 3. After extraction with methyl-tert-butyl ether (three times), the combined organic extracts are washed neutral, dried, and the solvent is spun off. The product that is obtained (15.82 g=61.1%) is used directly in the enol ether cleavage. To this end, the crude product is mixed with 330 ml of a 1 M sulfuric acid and 87 ml of glacial acetic acid and first stirred for two days at 90° C. Since the reaction was still not complete after two days, another 44 ml of glacial acetic acid was added. After a total of six days of stirring, the reaction mixture is made basic with solid potassium carbonate (pH 9, strong foaming) while being cooled in an ice batch, and it is extracted three times with methyl-tert-butyl ether. The ether phase is discarded after TLC monitoring. The aqueous phase is acidified with concentrated HCl up to a pH of 4. After the extraction with methyl-tert-butyl ether, washing and drying of the organic phase, the solvent is spun off, and the residue that is obtained (11.9 g=83.8%) is used in crude form in the next stage.

^{H-NMR (}300 MHz, CDCl₃): δ=1.09 (6H), 2.69 (2H), 2.84 (2H), 7.00 (1H), 7.13 (1H), 7.22 (2H).

### Ethyl-5-(3-chlorophenyl)-4,4-dimethyl-2-oxopentanoate

10.61 g (41.65 mmol) of 5-(3-chlorophenyl)-4,4-dimethyl-2-oxopentanoic acid is added in 259 ml of ethanol, mixed with 4.7 ml of concentrated sulfuric acid and refluxed for six and one-half hours. After cooling, the reaction mixture is poured into 500 ml of saturated sodium bicarbonate solution and extracted three times with 200 ml each of ethyl acetate. The combined organic extracts are washed with saturated sodium bicarbonate solution and brine. After drying on sodium sulfate, the solvent is spun off, and the residue is chromatographed on silica gel (mobile solvent ethyl acetate/hexane). 5.26 g (44.6%) of the desired ester is isolated.

¹H-NMR (300 MHz, CDCl₃): δ=1.05 (6H), 1.38 (3H), 2.68 (2H), 2.73 (2H), 4.30 (2H), 7.00 (1H), 7.12 (1H), 7.20 (2H).

### Ethyl-5-(3-chlorophenyl)-4,4-dimethyl-2-hydroxy-2-(trifluoromethyl)pentanoate

5.26 g (18.59 mmol) of ethyl-5-(3-chlorophenyl)-4,4-dimethyl-2-oxopentanoate is dissolved in 26.8 ml of THF and mixed with 3.17 g (22.3 mmol) of (trifluoromethyl)trimethylsilane. After 43 mg of tetrabutylammonium fluoride is added, the batch is stirred overnight at room temperature. 5.36 g of tetrabutylammonium fluoride is now added, and the reaction mixture is stirred for another two hours at room temperature. The reaction mixture is diluted with methyl-tert-butyl ether, and the phases are separated. After further shaking with methyl-tert-butyl ether, the combined organic extracts are washed with water and brine and dried. After the solvent is spun off, the residue is chromatographed on silica gel (mobile solvent ethyl acetate/hexane). 4.34 g (66.2%) of the desired product is isolated.

¹H-NMR (400 MHz, CDCl₃): δ=0.89 (3H), 1.00 (3H), 1.35 (3H), 1.90-2.06 (2H), 2.50 (1H), 2.80 (1H), 3.92 (1H), 4.27-4.45 (2H), 7.09 (1H), 7.20 (3H).

### 5-(3-Chlorophenyl)-2-hydroxy-4,4-dimethyl-2-(trifluoromethyl)pentanal

2 g (5.67 mmol) of ethyl-5-(3-chlorophenyl)-4,4-dimethyl-2-hydroxy-2-(trifluoromethyl)-pentanoate is dissolved in 74.1 ml of diethyl ether. At 0° C., 161.4 mg (4.25 mmol) of lithium aluminum hydride is added in portions. After one hour of stirring at 0° C., 12 ml of saturated sodium bicarbonate solution is added in drops, and the batch is stirred vigorously at room temperature for another two hours. After some silica gel is added, it is stirred for another 10 minutes, and the reaction mixture is then suctioned off via a glass fiber filter. The ether phase is washed with brine, dried on sodium sulfate, and the solvent is spun off. The crude product is chromatographed on a Flashmaster. 1.43 g (81.6%) of a 9:7 mixture that consists of the desired aldehyde and the starting material is obtained. This mixture is incorporated without further purification into the next stage.

### 5-(3-Chlorophenyl)-1,1,1-trifluoro-2-[(]1H-indazol-4-ylimino)-methyl]-4,4-dimethylpentan-2-ol

300 mg (0.97 mmol) of the previously described mixture that consists of aldehyde and ester and 129.39 mg (0.97 mmol) of 4-aminoindazole are mixed with 1.4 ml of glacial acetic acid and stirred for four days at room temperature. The reaction mixture is drawn off three times with toluene in a rotary evaporator, and the residue is then chromatographed on silica gel (mobile solvent ethyl acetate/hexane). 117.4 mg (28.5%) of the desired compound is isolated.

¹H-NMR (300 MHz, CDCl₃): δ=0.95 (3H), 1.09 (3H), 1.90 (1H), 2.24 (1H), 2.59 (1H), 2.85 (1H), 6.82 (1H), 7.09 (1H), 7.15-7.32 (3H), 7.38-7.52 (2H), 8.13 (1H), 8.20 (1H).

### 2-Chloro-6,7,8,9-tetrahydro-5-(1H-indazol-4-ylamino)-8,8-dimethyl-6-(trifluoromethyl)-5H-benzocyclohepten-6-ol

116 mg (0.27 mmol) of 5-(3-chlorophenyl)-1,1,1-trifluoro-2-[(1H-indazol-4-ylimino)-methyl]-4,4-dimethylpentan-2-ol is mixed with 5.47 ml of boron tribromide (1 M in dichloromethane) and then stirred for seven days at room temperature. The reaction mixture is then poured into a mixture that consists of saturated sodium bicarbonate solution and ice, and after the ethyl acetate is added, it is stirred vigorously for 20 minutes at room temperature. The ethyl acetate phase is separated, and the aqueous phase is extracted another time with ethyl acetate. The combined organic extracts are washed with water and with brine and dried. After the solvent is spun off, the residue is chromatographed on silica gel (mobile solvent (dichloromethane/methanol). 67.9 mg (58.5%) of the desired compound is isolated.

¹H-NMR (300 MHz, CD₃OD): δ=0.88 (3H), 1.19 (3H), 1.42 (1H), 1.90 (1H), 2.60 (1H), 3.11 (1H), 5.23 (1H), 5.73 (1H), 6.73 (1H), 7.00 (1H), 7.10 (1H), 7.18 (1H), 7.30 (1H), 8.09 (1H).

### EXAMPLE 4

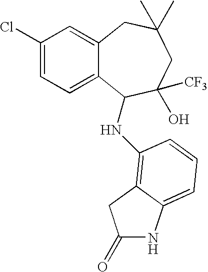

### 4-{[2-Chloro-6,7,8,9-tetrahydro-6-hydroxy-8,8-dimethyl-6-(trifluoromethyl)-5H-benzocyclohepten-5-yl]amino}-1,3-dihydro-2H-indol-2-one

### 7-{[5-(3-Chlorophenyl)-2-hydroxy-4,4-dimethyl-2-(trifluoromethyl)-pentylidene]amino}-1,3-dihydro-2H-indol-2-one

450 mg (1.46 mmol) of the mixture, described in Example 3, that consists of 5-(3-chlorophenyl)-2-hydroxy-4,4-dimethyl-2-(trifluoromethyl)-pentanal and the corresponding esters are stirred for four days at room temperature with 216 mg (1.46 mmol) of 4-amino-1,3-dihydro-2H-indol-2-one and 2.1 ml of glacial acetic acid. The reaction mixture is drawn off three times with toluene in a rotary evaporator, and the remaining residue is chromatographed on silica gel (mobile solvent hexane/ethyl acetate). 307.1 mg (48%) of the desired imine is isolated.

¹H-NMR (300 MHz, CDCl₃): δ=0.90 (3H), 1.02 (3H), 1.85 (1H), 2.20 (1H), 2.52 (1H), 2.81 (1H), 3.54 (2H), 4.90 (1H), 6.69 (1H), 6.86 (1H), 7.08 (1H), 7.15-7.32 (4H), 8.04 (1H), 8.50 (1H).

### 4-{[2-Chloro-6,7,8,9-tetrahydro-6-hydroxy-8,8-dimethyl-6-(trifluoromethyl)-5H-benzocyclohepten-5-yl]amino}-1,3-dihydro-2H-indol-2-one

305 mg (0.69 mmol) of the above-described imine is mixed at room temperature with 13.9 ml of a 1 M boron tribromide solution in dichloromethane and stirred for five days at this temperature. For working-up, the reaction mixture is poured into a mixture that consists of saturated sodium bicarbonate solution and ice. After dilution with 50 ml of ethyl acetate, the mixture is stirred vigorously for 20 minutes. The organic phase is separated, and the aqueous phase is shaken another time with 50 ml of ethyl acetate. The combined organic extracts are washed with water and brine and dried on sodium sulfate. After the desiccant is filtered off, and after the solvent is spun off, the remaining residue is chromatographed several times on silica gel (mobile solvent dichloromethane/methanol). 175.5 mg (57.5%) of the desired compound 4-{[2-chloro-6,7,8,9-tetrahydro-6-hydroxy-8,8-dimethyl-6-(trifluoromethyl)-5H-benzocyclohepten-5-yl]amino}-1,3-dihydro-2H-indol-2-one is isolated.

¹H-NMR (300 MHz, CD₃OD): δ=0.84 (3H), 1.15 (3H), 1.40 (1H), 1.85 (1H), 2.58 (1H), 3.06 (1H), 3.25 (1H, signal is less than the methanol signal), 3.45 (1H), 5.10 (1H), 5.90 (1H), 6.23 (1H), 6.89 (1H), 7.15 (2H), 7.28 (1H).

### EXAMPLE 5

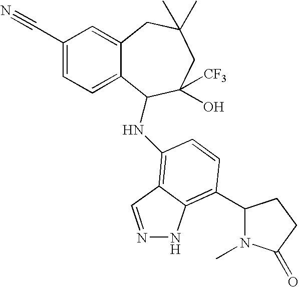

### 6,7,8,9-Tetrahydro-6-hydroxy-8,8-dimethyl-5-{[7-(1-methyl-5-oxopyrrolidin-2-yl)-1H-indazol-4-yl]amino}-6-(trifluoromethyl)-5H-benzocycloheptene-2-carbonitrile

35 mg (0.083 mmol) of the 2-chloro-6,7,8,9-tetrahydro-5-(1H-indazol-4-ylamino)-8,8-dimethyl-6-(trifluoromethyl)-5H-benzocyclohepten-6-ol, described in Example 3, is mixed together with 8.09 mg (0.17 mmol) of sodium cyanide and 18.04 mg (0.083 mmol) of nickel bromide in 1 ml of 1-methyl-2-pyrrolidinone and brought to reaction in a microwave (20 bar, 200° C., 20 minutes). For working-up, the reaction mixture is mixed with 5 ml of ethyl acetate. After two ml of water is added, the mixture is stirred vigorously for 15 minutes at room temperature. It is shaken another time with ethyl acetate. The combined organic extracts are washed with water and brine. After the organic phase is dried, the solvent is spun off, and the residue is chromatographed on a Flashmaster (amine phase, mobile solvent methanol/dichloromethane). 16.4 mg (38.8%) of 6,7,8,9-tetrahydro-6-hydroxy-8,8-dimethyl-5-{[7-(1-methyl-5-oxopyrrolidin-2-yl)-1H-indazol-4-yl]amino}-6-(trifluoromethyl)-5H-benzocycloheptene-2-carbonitrile (80%) is obtained.

¹H-NMR (300 MHz, CD₃OD): δ=0.85 (3H), 1.20 (3H), 1.45 (1H), 1.90-2.08 (2H), 2.32-2.60 (6H), 2.69 (1H), 3.22 (1H), 5.34 (1H), 5.75 (1H), 6.83 (1H), 7.45-7.59 (3H), 8.20 (1H).

### EXAMPLE 6

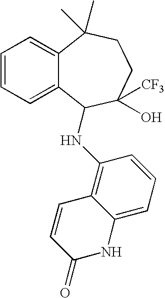

### (5R*-cis)-5-{[6,7,8,9-Tetrahydro-6-hydroxy-9,9-dimethyl-6-(trifluoromethyl)-5H-benzocyclohepten-5-yl]amino}quinolin-2(1H)-one

### 2-(2-Chlorophenyl)-2-methylpropanenitrile

25 g (164.92 mmol) of 2-chlorobenzyl cyanide is dissolved in 250 ml of dimethylformamide, and the solution is mixed with 68 g (346.33 mmol) of iodomethane. At 0° C., 13.9 g (346.33 mmol) of a sodium hydride suspension (w=55-60%) is now added in portions. After stirring overnight at room temperature, the reaction mixture is poured into ice water and shaken three times with methyl-tert-butyl ether. The combined organic extracts are washed with water and then with brine. After drying on sodium sulfate, the solvent is spun off and the residue is chromatographed on silica gel (mobile solvent methyl-tert-butyl ether/hexane). 27.25 g (91.8%) of the desired compound is isolated.

¹H-NMR (300 MHz, CDCl₃): δ=1.99 (6H), 7.24-7.33 (2H), 7.40-7.50 (2H).

### 2-(2-Chlorophenyl)-2-methylpropanal

27.25 g (151.69 mmol) of the above-described nitrile is dissolved in 510 ml of toluene. At −70° C., 186.9 ml of a 1.2 molar solution of DIBAH in toluene is added in drops. After two and one-half hours of stirring at this temperature, 26.1 ml of isopropanol and then 1.72 l of a 10% L-(+)-tartaric-acid solution are added in drops. In this case, the temperature increases. Altogether, the batch is stirred vigorously for one hour at room temperature. After three cycles of extraction with methyl-tert-butyl ether, the combined, organic extracts are washed with brine, dried and the solvent is spun off. The residue that is obtained (27.5 g=100%) is further incorporated in crude form.

¹H-NMR (300 MHz, CDCl₃): δ=1.50 (6H), 7.21-7.46 (4H), 9.72 (1H).

### 1-Chloro-2-(3,3-dibromo-1,1-dimethylprop-2-enyl)benzene

29.5 g (112.31 mmol) of triphenylphosphine is dissolved in 350 ml of dichloromethane. After 7.34 g (112.31 mmol) of zinc dust is added, the batch is cooled to 0° C., and then 37.2 g (112.31 mmol) of tetrabromomethane is added in portions. After 16 hours of stirring at room temperature, 10 g (54.75 mmol) of 2-(2-chlorophenyl)-2-methylpropanal, dissolved in 140 ml of dichloromethane, is added in drops. After two hours of stirring, the reaction mixture is mixed with 350 ml of hexane, stirred vigorously and suctioned off via a silica-gel-filled frit. After the residue is washed with 600 ml of hexane/dichloromethane (1:1), the filtrate is evaporated to the dry state. The residue is chromatographed on silica gel (mobile solvent ethyl acetate/hexane). 6.31 g, which turned out to be starting material, however, is isolated and was therefore used again in the reaction to form the double bond.

In this respect, in a modified variant, 34.3 g (103.46 mmol) of tetrabromomethane in 414 ml of dichloromethane is dissolved and mixed in portions at 0° C. with 54.4 g (207.41 mmol) of triphenylphosphine. After one hour of stirring at 0° C., the recovered 6.31 g (34.55 mmol) of 2-(2-chlorophenyl)-2-methylpropanol, dissolved in 80 ml of dichloromethane, is added in drops. The batch is stirred overnight at room temperature. The reaction mixture is poured into 1.3 liters of hexane. After one hour of vigorous stirring, it is suctioned off via Celite, and the solvent is spun off. The residue is chromatographed on silica gel (mobile solvent ethyl acetate/hexane). 4.67 g (41.4%) of the desired product (still contains some starting material) is isolated.

¹H-NMR (300 MHz, CDCl₃): δ=1.60 (6H), 6.93 (1H), 7.16-7.25 (2H), 7.38 (1H), 7.43 (1H).

### 1-Chloro-2-(1,1-dimethylprop-2-inyl)benzene

4.46 g (13.66 mmol) of the above-described dibromoalkene is dissolved in 180 ml of tetrahydrofuran and mixed at −70° C. drop by drop with 16.1 ml (26.8 mmol) of a butyllithium solution (15% in hexane). After two hours of stirring at this temperature, 2.7 ml of water is carefully added in drops, and the mixture is then stirred vigorously for two hours at room temperature. After the reaction mixture is mixed with methyl-tert-butyl ether, the organic phase is washed with water and then with brine. After the organic phase is dried, the solvent is spun off, and the residue (2.83 g>100%) is incorporated in crude form into the next stage.

¹H-NMR (500 MHz, CDCl₃): δ=1.71 (6H), 2.30 (1H), 7.16-7.20 (2H), 7.30 (1H), 7.63 (1H).

### Ethyl 5-(2-chlorophenyl)-2-hydroxy-5-methyl-2-(trifluoromethyl)hex-3-inoate

2.83 g (15.84 mmol) of 1-chloro-2-(1,1-dimethylprop-2-inyl)benzene is dissolved in 150 ml of tetrahydrofuran, and the mixture is cooled to −60° C. At this temperature, 9.8 ml (15.84 mmol) of n-butyllithium solution (15% in hexane) is now added in drops, and the batch is then stirred for two hours at 0° C. After repeated cooling to −60° C., 1.9 ml (15.84 mmol) of ethyl trifluoropyruvate is now added in drops. After 24 hours of stirring at room temperature, the reaction mixture is poured into saturated ammonium chloride solution. After 3 cycles of extraction with methyl-tert-butyl ether, the combined organic extracts are washed with brine. After drying with sodium sulfate and after the solvent is spun off, the residue is chromatographed on a Flashmaster. 2.4 g (43.6%) of the desired compound is isolated.

¹H-NMR (300 MHz, CDCl₃): δ=1.49 (3H), 1.79 (6H), 4.12 (1H), 4.41 (2H), 7.17-7.25 (2H), 7.39 (1H), 7.58 (1H).

### Ethyl 2-hydroxy-5-methyl-5-phenyl-2-(trifluoromethyl)hexanoate

2.3 g (6.59 mmol) is mixed with 231 mg of Pd/C (10%) and 16 ml of ethanol and stirred under a hydrogen atmosphere overnight at room temperature. The catalyst is suctioned off via a glass-pleated filter, and the filtrate is spun in. 2.14 g of a mixture that consists of the desired saturated product and the corresponding alkene compound is isolated.

This mixture is subjected another time to a hydrogenation (15 ml of ethanol, 214 mg of Pd/C, hydrogen atmosphere, 24 hours of stirring at room temperature). After the catalyst is suctioned off via a glass-fiber filter, the filtrate is spun in, and the residue is chromatographed on a Flashmaster. 995.2 mg (46.2%) of a product that primarily contains the dechlorine compound is isolated.

MS (Cl) m/e (relative intensity): 336 (100%)

### 2-Hydroxy-5-methyl-5-phenyl-2-(trifluoromethyl)hexanal

995.2 mg (2.82 mmol) of ethyl 2-hydroxy-5-methyl-5-phenyl-2-(trifluoromethyl)hexanoate is dissolved in 11.5 ml of diethyl ether and mixed at 0° C. in portions with 82.79 mg of lithium aluminum hydride. After one hour of stirring at this temperature, 10 ml of saturated sodium bicarbonate solution is carefully added in drops, and the batch is then stirred vigorously for one hour. After 3 cycles of extraction with methyl-tert-butyl ether, the combined organic extracts are washed with brine, dried with sodium sulfate, and the solvent is spun off. The remaining residue is chromatographed on a Flashmaster. 211.3 mg of the desired aldehyde is obtained.

¹H-NMR (400 MHz, CDCl₃): δ=1.25-1.38 (1H), 1.32 (6H), 1.60-1.85 (3H), 3.71 (1H), 7.15-7.40 (5H), 9.49 (1H).

### 5-{[4-(2-Hydroxy-5-methyl-5-phenyl-2-(trifluoromethyl)hexylidene]amino}quinolin-2(1H)-one

211 mg (0.684 mmol) of the above-described aldehyde and 109.8 mg (0.684 mmol) of 5-amino-quinolin-2(1H)-one are stirred in 2.6 ml of glacial acetic acid for three days at room temperature. The batch is spun in until a dry state is reached, and the residue is chromatographed on a Flashmaster. 177.9 mg (57.7%) of the desired imine is isolated.

¹H-NMR (400 MHz, CDCl₃): δ=1.30-1.40 (6H), 1.41-2.00 (4H), 4.70 (1H), 6.68-6.80 (2H), 7.13-7.25 (1H), 7.25-7.35 (4H), 7.40 (1H), 7.52 (1H), 7.82 (1H), 8.11 (1H).

### (5R *-cis)-5-{[6,7,8,9-Tetrahydro-6-hydroxy-9,9-dimethyl-6-(trifluoromethyl)-5H-benzocyclohepten-5-yl]amino}quinolin-2(1H)-one

83.85 mg (0.19 mmol) of the just-described imine is dissolved in one milliliter of dichloromethane. After cooling to 0° C, 1.9 ml of a one-molar solution of boron tribromide is added in drops in dichloromethane. The batch is then stirred for 14 days at room temperature. The reaction mixture is poured into a mixture that consists of saturated sodium bicarbonate solution and ice and extracted three times with ethyl acetate. The combined organic extracts are washed with brine, dried on sodium sulfate, and the solvent is spun off. The remaining residue is chromatographed on a Flashmaster. 10.8 mg (13%) of the desired compound is isolated.

¹H-NMR (400 MHz, DMSO [d6]): δ=1.28-1.50 (7H), 1.73-1.87 (1H), 1.89-2.01 (1H), 2.10-2.23 (1H), 5.14 (1H) 5.72 (1H), 6.20-6.55 (3H), 7.00-7.12 (2H), 7.20 (1H), 7.30 (1H), 7.40 (1H), 8.19 (1H), 11.5 (1H).

With use of the corresponding imines, the following could be synthesized analogously:

5-{[6,7,8,9-Tetrahydro-6-hydroxy-9,9-dimethyl-6-(trifluoromethyl)-5H-benzocyclohepten-5-yl]amino}isoquinolin-1(2H)-one;

5-{[6,7,8,9-Tetrahydro-6-hydroxy-9,9-dimethyl-6-(trifluoromethyl)-5H-benzocyclohepten-5-yl]amino}1,3-dihydro-2H-indol-2-one;

5-{[6,7,8,9-Tetrahydro-6-hydroxy-9,9-dimethyl-6-(trifluoromethyl)-5H-benzocyclohepten-5-yl]amino}2,3-dihydro-isoindol-1-one;

5-{[6,7,8,9-Tetrahydro-6-hydroxy-9,9-dimethyl-6-(trifluoromethyl)-5H-benzocyclohepten-5-yl]amino}isochromen-1-one;

9,9-Dimethyl-5-[(7,8-difluoro-2-methylquinazolin-5-yl)amino]-6-(trifluoromethyl)-6,7,8,9-tetrahydro-5H-benzocyclohepten-6-ol;

9,9-Dimethyl-5-[(2-methylquinazolin-5-yl)amino]-6-(trifluoromethyl)-6,7,8,9-tetrahydro-5H-benzocyclohepten-6-ol;

9,9-Dimethyl-5-[(2-methylquinolin-5-yl)amino]-6-(trifluoromethyl)-6,7,8,9-tetrahydro-5H-benzocyclohepten-6-ol;

5-{[1-Chloro-6,7,8,9-tetrahydro-6-hydroxy-9,9-dimethyl-6-(trifluoromethyl)-5H-benzocyclohepten-5-yl]amino}quinolin-2(1H)-one;

1-Chloro-9,9-dimethyl-5-[(2-methylquinazolin-5-yl)amino]-6-(trifluoromethyl)-6,7,8,9-tetrahydro-5H-benzocyclohepten-6-ol;

5-{[2-Chloro-6,7,8,9-tetrahydro-6-hydroxy-9,9-dimethyl-6-(trifluoromethyl)-5H-benzocyclohepten-5-yl]amino}quinolin-2(1H)-one;

2-Chloro-9,9-dimethyl-5-[(2-methylquinazolin-5-yl)amino]-6-(trifluoromethyl)-6,7,8,9-tetrahydro-5H-benzocyclohepten-6-ol;

5-{[1-Chloro-2-fluoro-6,7,8,9-tetrahydro-6-hydroxy-9,9-dimethyl-6-(trifluoromethyl)-5H-benzocyclohepten-5-yl]amino}quinolin-2(1H)-one;

1-Chloro-2-fluoro-9,9-dimethyl-5-[(2-methylquinazolin-5-yl)amino]-6-(trifluoromethyl)-6,7,8,9-tetrahydro-5H-benzocyclohepten-6-ol;

5-{[2-Chloro-1-fluoro-6,7,8,9-tetrahydro-6-hydroxy-9,9-dimethyl-6-(trifluoromethyl)-5H-benzocyclohepten-5-yl]amino}quinolin-2(1H)-one;

2-Chloro-1-fluoro-9,9-dimethyl-5-[(2-methylquinazolin-5-yl)amino]-6-(trifluoromethyl)-6,7,8,9-tetrahydro-5H-benzocyclohepten-6-ol;

5-{[1-Bromo-6,7,8,9-tetrahydro-6-hydroxy-9,9-dimethyl-6-(trifluoromethyl)-5H-benzocyclohepten-5-yl]amino}quinolin-2(1H)-one;

1-Bromo-9,9-dimethyl-5-[(2-methylquinazolin-5-yl)amino]-6-(trifluoromethyl)-6,7,8,9-tetrahydro-5H-benzocyclohepten-6-ol;

5-{[2-Bromo-6,7,8,9-tetrahydro-6-hydroxy-9,9-dimethyl-6-(trifluoromethyl)-5H-benzocyclohepten-5-yl]amino}quinolin-2(1H)-one;

2-Bromo-9,9-dimethyl-5-[(2-methylquinazolin-5-yl)amino]-6-(trifluoromethyl)-6,7,8,9-tetrahydro-5H-benzocyclohepten-6-ol;

5-{[1-Bromo-2-chloro-6,7,8,9-tetrahydro-6-hydroxy-9,9-dimethyl-6-(trifluoromethyl)-5H-benzocyclohepten-5-yl]amino}quinolin-2(1H)-one;

1-Bromo-2-chloro-9,9-dimethyl-5-[(2-methylquinazolin-5-yl)amino]-6-(trifluoromethyl)-6,7,8,9-tetrahydro-5H-benzocyclohepten-6-ol;

5-{[2-Bromo-1-chloro-6,7,8,9-tetrahydro-6-hydroxy-9,9-dimethyl-6-(trifluoromethyl)-5H-benzocyclohepten-5-yl]amino}quinolin-2-(1H)-one;

2-Bromo-1-chloro-9,9-dimethyl-5-[(2-methylquinazolin-5-yl)amino]-6-(trifluoromethyl)-6,7,8,9-tetrahydro-5H-benzocyclohepten-6-ol;

5-{[1,2-Dichloro-6,7,8,9-tetrahydro-6-hydroxy-9,9-dimethyl-6-(trifluoromethyl)-5H-benzocyclohepten-5-yl]amino}quinolin-2(1H)-one;

1,2-Dichloro-9,9-dimethyl-5-[(2-methylquinazolin-5-yl)amino]-6-(trifluoromethyl)-6,7,8,9-tetrahydro-5H-benzocyclohepten-6-ol.

Without further elaboration, it is believed that one skilled in the art can, using the preceding description, utilize the present invention to its fullest extent. The following preferred specific embodiments are, therefore, to be construed as merely illustrative, and not limitative of the remainder of the disclosure in any way whatsoever.

In the foregoing and in the following examples, all temperatures are set forth uncorrected in degrees Celsius, and all parts and percentages are by weight, unless otherwise indicated.

The entire disclosures of all applications, patents and publications, cited herein and of corresponding German Application No. 102005014090.4, filed Mar. 22, 2005, and U.S. Provisional Application Ser. No. 60/669,885, filed Apr. 11, 2005, are incorporated by reference herein.

The preceding examples can be repeated with similar success by substituting the generically or specifically described reactants and/or operating conditions of this invention for those used in the preceding examples.

From the foregoing description, one skilled in the art can easily ascertain the essential characteristics of this invention and, without departing from the spirit and scope thereof, can make various changes and modifications of the invention to adapt it to various usages and conditions.

1. Compounds of general formula (I), 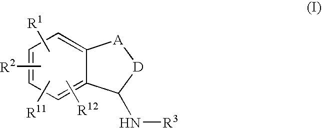
in which
R¹ and R², independently of one another, mean a hydrogen atom, a hydroxy group, a halogen atom, an optionally substituted (C₁-C₁₀)-alkyl group, a (C₁-C₁₀)-alkoxy group, a (C₁-C₁₀)-alkylthio group, a (C₁-C₅)-perfluoro-alkyl group, a cyano group, or a nitro group, or R¹ and R² together mean a group that is selected from the groups —O—(CH₂)ₙ—O—, —O—(CH₂)ₙ—CH₂—, —O—CH═CH—, —(CH₂)ₙ₊₂—, —NH—(CH₂)ₙ₊₁—, —N(C₁-C₃-alkyl)-(CH₂)ₙ₊₁—, and —NH—N═CH—,
whereby n=1 or 2, and the terminal atoms are linked to directly adjacent ring-carbon atoms, or NR⁸R⁹,
whereby R⁸ and R⁹, independently of one another, can be hydrogen, C₁-C₅-alkyl or (CO)—C₁-C₅-alkyl,
R¹¹ means a hydrogen atom, a hydroxy group, a halogen atom, a cyano group, an optionally substituted (C₁-C₁₀)-alkyl group, a (C₁-C₁₀)-alkoxy group, a (C₁-C₁₀)-alkylthio group, or a (C₁-C₅)-perfluoroalkyl group,
R¹² means a hydrogen atom, a hydroxy group, a halogen atom, a cyano group, an optionally substituted (C₁-C₁₀)-alkyl group, or a (C₁-C₁₀)-alkoxy group,
R³ means a C₁-C₁₀-alkyl group, which optionally can be substituted by a group that is selected from 1-3 hydroxy groups, halogen atoms, or 1-3 (C₁-C₅)-alkoxy groups,
an optionally substituted (C₃-C₇)-cycloalkyl group,
an optionally substituted heterocyclyl group,
an optionally substituted aryl group,
a monocyclic or bicyclic heteroaryl group that optionally is substituted by one or more groups selected from (C₁-C₅)-alkyl groups (which optionally themselves can be substituted by 1-3 hydroxy groups or 1-3 COOR¹⁰ groups, whereby R¹⁰ means any hydroxy protective group, a benzyl group or a C₁-C₁₀-alkyl group), (C₁-C₅)-alkoxy groups, halogen atoms, or exomethylene groups and that optionally contains 1-3 nitrogen atoms and/or 1-2 oxygen atoms and/or 1-2 sulfur atoms and/or 1-2 keto groups, whereby these groups can be linked via any position to the amine of the 5H-benzocycloheptene system and optionally can be hydrogenated at one or more sites,
A means a —CR⁶R⁷—CH₂— group or a —CH₂—CR⁶R⁷— group,
D means a —CR⁴R⁵—CH₂— group or a —CH₂—CR⁴R⁵— group,
R⁴ means a hydroxy group, a group OR¹⁰ or an O(CO)R¹⁰ group,
R⁵ means a (C₁-C₅)-alkyl group or an optionally partially or completely fluorinated (C₁-C₅)-alkyl group, a (C₃-C₇)cycloalkyl group, a (C₁-C₈)alkylene(C₃-C₇)cycloalkyl group, a (C₂-C₈)alkenylene(C₃-C₇)cyclo-alkyl group, a heterocyclyl group, a (C₁-C₈)alkyleneheterocyclyl group, a (C₂-C₈)alkenyleneheterocyclyl group, an aryl group, a (C₁-C₈)alkylenaryl group, a (C₂-C₈)alkenylenaryl group, a (C₂-C₈)alkinylenaryl group, a monocyclic or bicyclic heteroaryl group that optionally is substituted by 1-2 keto groups, 1-2 (C₁-C₅)-alkyl groups, 1-2 (C₁-C₅)-alkoxy groups, 1-3 halogen atoms, or 1-2 exomethylene groups and that contains 1-3 nitrogen atoms and/or 1-2 oxygen atoms and/or 1-2 sulfur atoms; a (C₁-C₈)alkyleneheteroaryl group or a (C₂-C₈)alkenyleneheteroaryl group, whereby these groups can be linked via any position to the 5H-benzocycloheptene system and optionally can be hydrogenated at one or more sites,
R⁶ and R⁷, independently of one another, mean a hydrogen atom, a methyl or ethyl group or, together with the carbon atom of the 5H-benzocycloheptene system, a (C₃-C₆)-cycloalkyl ring.

2. Stereoisomers of general formula (I) according to claim 1, 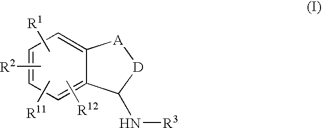
in which
R¹ and R², independently of one another, mean a hydrogen atom, a hydroxy group, a halogen atom, a (C₁-C₁₀)-alkyl group, a (C₁-C₁₀)-alkoxy group, a (C₁-C₁₀)-alkylthio group, a (C₁-C₅)-perfluoroalkyl group, a cyano group, or a nitro group, or R¹ and R² together mean a group that is selected from the groups —O—(CH₂)ₙ—O—, —O—(CH₂)ₙ—CH₂—, —O—CH═CH—, or —(CH₂)ₙ₊₂—, whereby n=1 or 2, and the terminal atoms are linked to directly adjacent ring-carbon atoms, or NR⁸R⁹,
whereby R⁸ and R⁹, independently of one another, can be hydrogen, C₁-C₅-alkyl or (CO)—C₁-C₅-alkyl,
R¹¹ and R¹² mean a hydrogen atom,
R³ means a C₁-C₁₀-alkyl group, which optionally can be substituted by a group that is selected from 1-3 hydroxy groups, halogen atoms, or 1-3 (C₁-C₅)-alkoxy groups,
an optionally substituted phenyl group or a naphthyl group,
a monocyclic or bicyclic heteroaryl group that optionally is substituted by 1-2 keto groups, 1-2 (C₁-C₅)-alkyl groups, 1-2 (C₁-C₅)-alkoxy groups, 1-3 halogen atoms, or 1-2 exomethylene groups and that contains 1-3 nitrogen atoms and/or 1-2 oxygen atoms and/or 1-2 sulfur atoms, whereby these groups can be linked via any position to the amine of the benzocycloheptene system and optionally can be hydrogenated at one or more sites,
A means a —CR⁶R⁷—CH₂— group or a —CH₂—CR⁶R⁷— group,
D means a —CR⁴R⁵—CH₂— group or a —CH₂—CR⁴R⁵— group,
R⁴ means a hydroxy group,
R⁵ means a (C₁-C₅)-alkyl group or an optionally partially or completely fluorinated (C₁-C₅)-alkyl group, an aryl group, a (C₁-C₈)alkylenaryl group, a (C₂-C₈)alkenylenaryl group, a (C₃-C₇)cycloalkyl group, a (C₁-C₈)alkylene(C₃-C₇)cycloalkyl group, or a (C₂-C₈)alkenylene(C₃-C₇)cycloalkyl group,
R⁶ and R⁷, independently of one another, mean a hydrogen atom, a methyl or ethyl group, or, together with the carbon atom of the benzocycloheptene system, a (C₃-C₆)-cycloalylring.

3. Stereoisomers of general formula (I) according to claim 1, 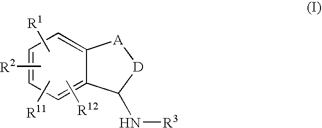
in which
R¹ and R², independently of one another, mean a hydrogen atom, a hydroxy group, a halogen atom, a (C₁-C₁₀)-alkyl group, a (C₁-C₁₀)-alkoxy group, a (C₁-C₁₀)-alkylthio group, a (C₁-C₅)-perfluoroalkyl group, a cyano group, or a nitro group, or R¹ and R² together mean a group that is selected from the groups —O—(CH₂)ₙ—O—, —O—(CH₂)ₙ—CH₂—, —O—CH═CH—, and —(CH₂)ₙ₊₂—, whereby n=1 or 2, and the terminal atoms are linked to directly adjacent ring-carbon atoms,
or NR⁸R⁹,
whereby R⁸ and R⁹, independently of one another, can be hydrogen, C₁-C₅-alkyl or (CO)—C₁-C₅-alkyl,
R¹¹ and R¹² mean a hydrogen atom,
R³ means a C₁-C₁₀-alkyl group, which optionally can be substituted by a group that is selected from 1-3 hydroxy groups, halogen atoms, or 1-3 (C₁-C₅)-alkoxy groups,
an optionally substituted phenyl group,
a monocyclic or bicyclic heteroaryl group that optionally is substituted by 1-2 keto groups, 1-2 (C₁-C₅)-alkyl groups, 1-2 (C₁-C₅)-alkoxy groups, 1-3 halogen atoms, or 1-2 exomethylene groups and that contains 1-3 nitrogen atoms and/or 1-2 oxygen atoms and/or 1-2 sulfur atoms, whereby these groups can be linked via any position to the amine of the benzocycloheptene system and optionally can be hydrogenated at one or more sites,
A means a —CR⁶R⁷—CH₂— group or a —CH₂—CR⁶R⁷— group,
D means a —CR⁴R⁵—CH₂— group or a —CH₂—CR⁴R⁵— group,
R⁴ means a hydroxy group,
R⁵ means a (C₁-C₅)-alkyl group or an optionally partially or completely fluorinated (C₁-C₅)-alkyl group, an aryl group, a (C₁-C₈)alkylenaryl group, a (C₂-C₈)alkenylenaryl group, a (C₃-C₇)cycloalkyl group, a (C₁-C₈)alkylene(C₃-C₇)cycloalkyl group, or a (C₂-C₈)alkenylene(C₃-C₇)cycloalkyl group,
R⁶ and R⁷, independently of one another, mean a hydrogen atom, a methyl or ethyl group, or, together with the carbon atom of the 5H-benzocycloheptene system, a (C₃-C₆)-cycloalkylring.

4. Stereoisomers of general formula (I) according to claim 1, 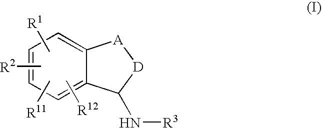
in which
R¹ and R², independently of one another, mean a hydrogen atom, a hydroxy group, a halogen atom, a (C₁-C₅)-alkyl group, a (C₁-C₅)-alkoxy group, a (C₁-C₅)-perfluoroalkyl group, or a cyano group, or R¹ and R² together mean a group that is selected from the groups —O—(CH₂)ₙ—O—, —O—(CH₂)ₙ—CH₂—, —O—CH═CH—, or —(CH₂)ₙ₊₂—,
whereby n=1 or 2, and the terminal atoms are linked to directly adjacent ring-carbon atoms,
R¹¹ and R¹² mean a hydrogen atom,
R³ means a C₁-C₁₀-alkyl group, which optionally can be substituted by 1-3 hydroxy groups, halogen atoms, a phenyl, phthalidyl, isoindolyl, dihydroindolyl, dihydroisoindolyl, dihydroisoquinolinyl, thiophthalidyl, benzoxazinonyl, phthalazinonyl, quinolinyl, isoquinolinyl, quinolonyl, isoquinolonyl, indazolyl, benzothiazolyl, quinazolinyl, quinoxalinyl, cinnolinyl, phthalazinyl, 1,7- or 1,8-naphthyridinyl, dihydroindolonyl, dihydroisoindolonyl, benzimidazolyl or indolyl group that optionally is substituted with C₁-C₅-alkyl, halogen, hydroxy, or C₁-C₅-alkoxy, whereby these groups can be linked via any position to the amine of the 5H-benzocycloheptene system and optionally can be substituted in one or more places with 1-2 keto groups, 1-2 (C₁-C₃)-alkyl groups, 1-2 (C₁-C₃)-alkoxy groups, 1-3 halogen atoms, or 1-2 exomethylene groups, and optionally can be hydrogenated at one or more sites,
A means a —CR⁶R⁷—CH₂— group or a —CH₂—CR⁶R⁷— group,
D means a —CR⁴R⁵—CH₂— group or a —CH₂—CR⁴R⁵— group,
R⁴ means a hydroxy group,
R⁵ means a (C₁-C₅)-alkyl group or an optionally partially or completely fluorinated (C₁-C₅)-alkyl group,
R⁶ and R⁷, independently of one another, mean a hydrogen atom, a methyl group or ethyl group, or, together with the carbon atom of the benzocycloheptene system, a (C₃-C₆)-cycloalkyl ring.

5. Stereoisomers of general formula (I) according to claim 1, 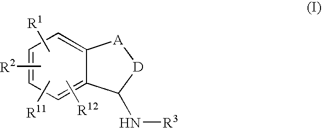
in which
R¹ and R², independently of one another, mean a hydrogen atom, a hydroxy group, a halogen atom, a (C₁-C₅)-alkyl group, a (C₁-C₅)-perfluoroalkyl group, a cyano group, a (C₁-C₅)-alkoxy group, or together a (C₁-C₂)-alkylenedioxy group, whereby then R¹ and R² must be directly adjacent,
R³ means a phenyl, phthalidyl, isoindolyl, dihydroindolyl, dihydroisoindolyl, dihydroisoquinolinyl, thiophthalidyl, benzoxazinonyl, phthalazinonyl, quinolinyl, isoquinolinyl, quinolonyl, isoquinolonyl, indazolyl, benzothiazolyl, quinazolinyl, quinoxalinyl, cinnolinyl, phthalazinyl, 1,7- or 1,8-naphthyridinyl, dihydroindolonyl, dihydroisoindolonyl, benzimidazolyl or indolyl group that optionally is substituted with C₁-C₅-alkyl, halogen, hydroxy, or C₁-C₅-alkoxy,
whereby these groups can be linked via any position to the amine of the benzocycloheptene system and optionally can be substituted in one or more places with 1-2 keto groups, 1-2 (C₁-C₃)-alkyl groups, 1-2 exomethylene groups and optionally can be hydrogenated at one or more sites,
A means a —CR⁶R⁷—CH₂— group or a —CH₂—CR⁶R⁷— group,
D means a —CR⁴R⁵—CH₂— group or a —CH₂—CR⁴R⁵— group,
R⁴ means a hydroxy group,
R⁵ means a (C₁-C₅)-alkyl group or an optionally partially or completely fluorinated (C₁-C₅)-alkyl group,
R⁶ and R⁷, independently of one another, mean a hydrogen atom, a methyl or ethyl group or, together with the carbon atom of the benzocycloheptene system, a (C₃-C₆)-cycloalkyl ring.

6. Stereoisomers of general formula (I) according to claim 1, in which
R¹ and R², independently of one another, mean a hydrogen atom, a hydroxy group, a halogen atom, a (C₁-C₃)-alkyl group, a CF₃ group, a cyano group, or a methoxy group,
R¹¹ and R¹² mean a hydrogen atom,
R³ means an isoindolyl, dihydroindolyl; dihydroisoindolyl, dihydroisoquinolinyl, quinolinyl, isoquinolinyl, quinazolinyl, quinolonyl, isoquinolonyl, indazolyl, dihydroindolonyl, or dihydroisoindolonyl group that optionally is substituted with C₁-C₃-alkyl, halogen, hydroxy, C₁-C₃-alkoxy, or methylpyrrolidin-2-on-5-yl, whereby these groups can be linked via any position to the amine of the benzocycloheptene system and optionally can be substituted in one or more places with 1-2 keto groups, 1-2 (C₁-C₃)-alkyl groups, or 1-2 exomethylene groups and optionally can be hydrogenated at one or more sites,
A means a —CR⁶R⁷—CH₂— group or a —CH₂—CR⁶R⁷— group,
D means a —CR⁴R⁵—CH₂— group or a —CH₂—CR⁴R⁵— group,
R⁴ means a hydroxy group,
R⁵ means a completely fluorinated (C₁-C₃)-alkyl group,
R⁶ and R⁷, independently of one another, mean a hydrogen atom, or a methyl or ethyl group.

7. Use of the stereoisomers according to one of the preceding claims for the production of a pharmaceutical agent.

8. Use of the stereoisomers of claims 1-5 for the production of a pharmaceutical agent for treating inflammatory diseases.

9. Use according to claim 8 for the production of a pharmaceutical agent for the treatment of
Lung diseases that are accompanied by inflammatory, allergic and/or proliferative processes,
Rheumatic diseases, autoimmune diseases, or joint diseases that are accompanied by inflammatory, allergic and/or proliferative processes,
Allergies or pseudoallergic diseases that are accompanied by inflammatory and/or proliferative processes,
Dermatological diseases that are accompanied by inflammatory, allergic and/or proliferative processes,
Eye diseases that are accompanied by inflammatory, allergic and/or proliferative processes,
Diseases of the ear, nose, and throat area that are accompanied by inflammatory, allergic and/or proliferative processes, or endocrine diseases that are accompanied by inflammatory, allergic and/or proliferative processes.

10. Pharmaceutical preparations that contain at least one stereoisomer according to claims 1-5 or mixtures thereof as well as pharmaceutically compatible vehicles.

11. Process for the production of stereoisomers of general formula I, characterized in that stereoisomers of general formula II 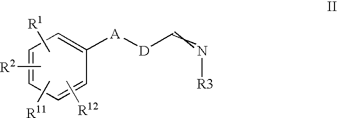
in which the radicals have the above-indicated meanings are cyclized with the addition of inorganic or organic acids or Lewis acids.

12. Process for the production of the compounds of general formula II according to claim 11, in which A-D means —CH₂—(CR⁶R⁷)—(CH₂)—C(OH)(CF₃)— (compound of general formula IIa), wherein a compound of general formula III, in which LG means chloride, bromide, iodide, sulfate, or sulfonate, is reacted under basic conditions in organic solvents with an aldehyde of general formula IV to compounds of general formula V under phase transfer catalysis with commonly used phase transfer catalysts to form compounds of general formula V, 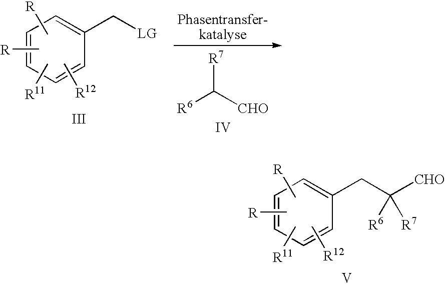
[Phase Transfer Catalysis]
the aldehyde V is then reacted under Horner-Wittig conditions with the reagent VI, whereby R^{x} means a (C₁-C₃)-alkyl group or a benzyl group, and the compound of general formula VII is obtained. After the ester is saponified according to the method known to one skilled in the art, 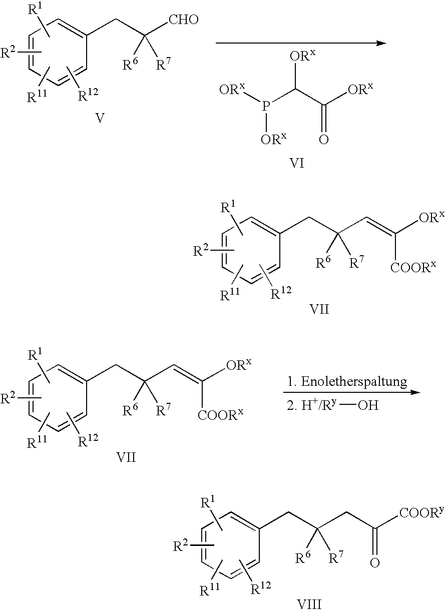
[Enol Ether Cleavage]
the enol ether is cleaved under acidic conditions, and the α-keto acid that is obtained is esterified under commonly used acidic conditions with an alcohol R^{y}—OH, whereby R^{y} means (C₁-C₅)-alkyl, to form an α-keto ester of general formula VIII, which is reacted with Ruppert's reagent, CF₃Si(CH₃)₃, 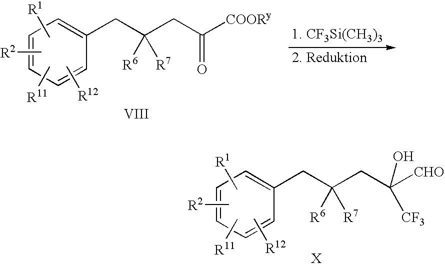
[Reduction]
and is reduced under the conditions known to one skilled in the art to form aldehyde X, which then—as already described in the prior art—is reacted with an amine of general formula R³—NH₂ to form the imine of general formula IIa, whereby R³ has the meaning that is indicated in the preceding claims, and A-D means —CH₂—(CR⁶R⁷)—(CH₂)—C(OH)(CF)₃—, which can be further reacted according to claim 11.

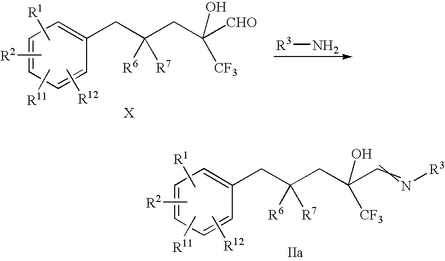

13. Process for the production of compounds of general formula II according to claim 11, A-D means —(CR⁶R⁷)—(CH₂)₂—C(OH)(CF₃)— (compound of general formula IIb), wherein a nitrile of general formula XI is optionally reacted in succession with sodium hydride and the corresponding alkyl halides R⁶-Hal and/or R⁷-Hal or Hal-[(C₂-C₅)-alkylene]-Hal to form a compound of general formula XII, 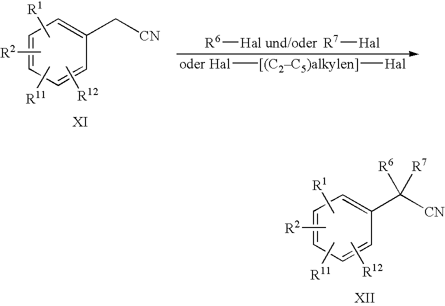
[R⁶-Hal and/or R⁷-Hal or Hal-[(C₂-C₅)alkylene]-Hal]
which under common conditions is reduced to form aldehyde 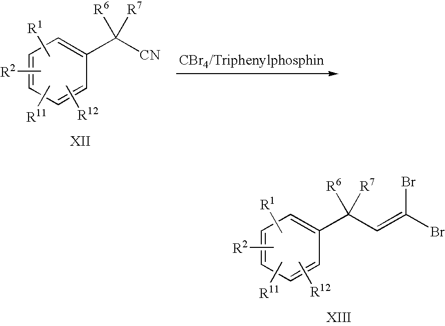
[ . . . Triphenylphosphine]
and then is reacted with CBr₄/triphenylphosphine optionally with the addition of zinc to form a compound of general formula XIII, 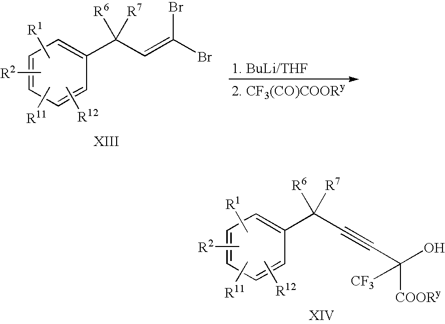
which is converted with butyllithium into acetylene and is converted with CF₃—(CO)COOR^{y} into the ester XIV. The ester XIV is hydrogenated according to methods known to one skilled in the art to form esters of general formula VIIIb and is reduced to aldehyde analogously to the process according to claim 12 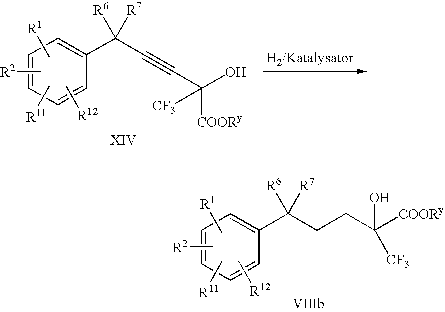
[H₂/Catalyst]
and is reacted with the amine R³—NH₂ to form the imine 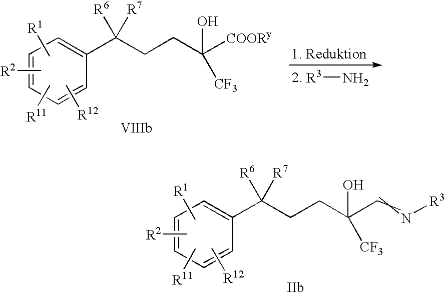
[Reduction]
of general formula IIb, whereby R³ has the meaning indicated in the preceding claims and in which A-D means —(CR⁶R⁷)—(CH₂)₂—C(OH)(CF₃)—, which can be further reacted according to claim 11.

14. Stereoisomers of general formula I, according to one of claims 1-5, in the form of salts with physiologically compatible anions.

15. Stereoisomers according to claim 14, in the form of their hydrochlorides, sulfates, nitrates, phosphates, pivalates, maleates, fumarates, tartrates, benzoates, mesylates, citrates or succinates.
